(19) United States
(12) Patent Application Publication
(10) Pub. No.: US 2013/0274470 A1
(43) Pub. Date: Oct. 17, 2013

(54) Triazolium Carbene Catalysts and Stereoselective Bond Forming Reactions Thereof

(71) Applicant: Colorado State University Research Foundation, Fort Collins, CO (US)

(72) Inventors: Tomislav Rovis, Fort Collins, CO (US); Harit U. Vora, Fort Collins, CO (US)

(21) Appl. No.: 13/912,765

(22) Filed: Jun. 7, 2013

Related U.S. Application Data
(62) Division of application No. 13/020,693, filed on Feb. 3, 2011.
(60) Provisional application No. 61/300,905, filed on Feb. 3, 2010.

(51) Int. Cl. B01J 31/02; C07B 41/08; C07D 333/24; C07D 498/04; C07C 51/16

(52) U.S. Cl. CPC B01J 31/0271; C07D 498/04; C07C 51/16; C07D 333/24; C07B 41/08
USPC 544/99; 562/418; 562/400; 549/79; 502/164

(57) ABSTRACT

Provided herein are triazolium carbine catalysts useful for asymmetric hydration, fluorination, and deuteration, and processes for their preparation. Also provided are synthetic reactions in which these catalysts are used, in particular, in stereoselective formation of carbon-chlorine, carbon-hydrogen, carbon-fluorine, and carbon-deuterium bonds.

### RELATED APPLICATIONS

This application is a divisional of U.S. patent application Ser. No. 13/020,693, entitled “Triazolium Carbene Catalysts and Stereoselective Bond Forming Reactions Thereof”, filed Feb. 3, 2011, which claims priority to U.S. Provisional Patent Application No. 61/300,905, entitled “N-heterocyclic Carbene Catalyzed Asymmetric Hydration: Direct Synthesis of Select Alpha-Proteo and Alpha-Deutero Carboxylic Acids”, filed Feb. 3, 2010, which is incorporated herein by reference in its entirety.

This invention was made with government support under R01 GM072586 awarded by National Institutes of Health. The government has certain rights in the invention.

### TECHNICAL FIELD

The present invention relates to triazolium carbene catalysts useful for asymmetric bond formation, and to processes for their preparation. In particular, the invention relates to use of these compounds in asymmetric synthesis of carbon-hydrogen, carbon-fluorine, carbon-chlorine, and carbon-deuterium formation. The catalysts are particularly useful when enantioselectivity is also required during the asymmetric bond formations.

### BACKGROUND OF THE INVENTION

Asymmetric carbon-hydrogen, carbon-deuteron, carbon-fluorine and carbon-chlorine bond formation remain a formidable challenge in organic synthesis arts. Recent advances in the field of asymmetric bond formation have been limited to specific substrates with limited target substitution patterns. These limitations are particularly prevalent when the product of the asymmetric reaction is an enantiomeric compound. These limitations have made it particularly difficult to produce target chemical agents useful in pharmaceutical drug formation.

The present invention provides novel catalysts for overcoming asymmetric bond formation on a wide array of substrates. The catalysts are relatively inexpensive, versatile and useful in providing enantioenriched products when compared to appropriate conventional methodologies.

As such, there is a need in the field to provide safe, reactive, less hazardous, cost effective, catalysts, especially catalysts capable of asymmetric hydration, fluorination, and deuteration.

The present invention is directed toward overcoming one or more of the problems discussed above.

### SUMMARY OF THE INVENTION

The present invention provides novel triazolium carbene catalysts (triazolium catalyst herein) for use in stereocontrolled introduction of hydrogen, deuterium, chlorine, or fluorine atoms into a variety of substrate molecules. The resulting compounds, i.e., compounds formed from reaction with an aldehyde containing substrate molecule, have been shown to have tremendous potential in medical, agricultural, plastics, and other like industries.

In general, triazolium catalysts of the invention are compounds of formula (I):

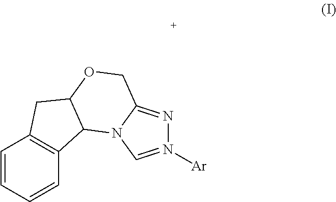

wherein Ar is an unsubstituted or substituted phenyl, naphthyl, pyridyl, pyrymidinyl, furyl, thiophenyl, pyrrolyl, or quinoline group, or any suitable heteroaromatic group. In some aspects, the Ar can be unsubstituted. In other aspects, the Ar is substituted with one or more electron-releasing or electron-withdrawing groups, for example, a substituent selected from the group consisting of X, RXₙ, RO, and NO₂, wherein R can be a substituted or unsubstituted branched or straight chain alkyl, X can be a halogen or pseudohalogen, and n is 1-3. Exemplary electron withdrawing groups include CH₃O, Cl, F, CF₃, NO₂ and CH₃.

Also provided herein are methods for asymmetric hydration, fluorination, chlorination, and deuteration of α-reducible aldehydes. One method comprises contacting the α-reducible aldehyde with a compound of formula (I). In some embodiments, a method for asymmetric hydration comprises contacting the α-reducible aldehyde with a proton donor and a compound of formula (I) forming the respective carboxylic acid of the enal. In some embodiments, a method for asymmetric deuteration comprises contacting the α-reducible aldehyde with D₂O and a compound of formula (I), wherein the α-reducible aldehyde incorporates an α-deuteron. An enal is an exemplary α-reducible aldehyde.

Further provided herein are methods for asymmetric hydration, fluorination, chlorination, and deuteration of drug analogs. The method comprises contacting a drug analog with a compound of formula (I). In some embodiments, a method for asymmetric hydration or deuteration comprises contacting the drug analog with a proton donor and a compound of formula (I). The drug analog can include at least one target aliphatic or functional group for asymmetric hydration to form the acid of the drug analog. In some aspects, the acid form of the drug analog is a chiral, non-racemic acid.

Still further provided are synthesis schemes for producing the catalysts described herein, as well as numerous Examples that illustrate the utility of all aspects of the invention.

These and various other features as well as advantages which characterize the invention will be apparent from a reading of the following detailed description and a review of the appended claims.

### DETAILED DESCRIPTION OF THE INVENTION

Provided herein is a novel class of tetracyclic triazolium carbene catalysts for catalytic asymmetric C—H, C—F, C—Cl, and C-D bond formation on a variety of useful substrates. This new class of catalyst is shown to facilitate high enantioselective control while participating in a variety of reactions in good yield. In some embodiments, potential substrates for this new class of catalyst include: α-reducible aldehydes, enals (α,β-unsaturated aldehydes), α-fluoro aldehydes, α,β-epoxyaldehydes, α,β-aziridinoaldehydes, α-halo aldehydes (including chloro, bromo, and iodo), α-acetoxy aldehydes, α-phenoxy aldehydes, and any other aldehyde that has a moderate leaving group at the alpha position. Sometimes the substrate is referred to as an activated aldehyde. In other embodiments, strained rings act as substrates (including, for example, cyclopropanes with carbon as a leaving group) for the catalysts herein.

The catalysts of the invention are unexpectedly versatile and capable of providing efficient yields in an asymmetric manner. The catalysts and reaction components are relatively inexpensive compared to like reactions with conventional methodologies. Finally, these catalysts herein have significant capacity for turnover, thereby providing excellent catalytic activity and product yield, and allow for superior enantioenriched product formation, a significant advancement in the art.

### The Catalysts

In general, the invention provides compounds of formula (I):

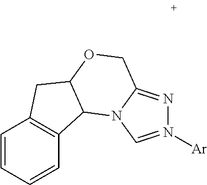

in which
Ar is selected from (i) phenyl group (Ph); (ii) naphthyl; (iii) pyridyl; (iv) pyrymidinyl; (v) furyl; (vi) thiophene (vii) pyrrolyl; (viii) quinoline; and (ix) any suitable heteroaromatic. Each group (i-ix) can be unsubstituted or substituted. Ar can be substituted with one or more electron-releasing or electron-withdrawing groups, for example, a substituent selected from the group consisting of X, RXₙ, RO, and NO₂, wherein R can be a substituted or unsubstituted branched or straight chain alkyl, X can be a halogen or pseudohalogen, and n is 1-3. Exemplary electron withdrawing groups include CH₃O, Cl, F, CF₃, NO₂ and CH₃.

Thus, the Ar of formula (I) can be very broad in scope with electron releasing and electron withdrawing substitution that varies widely. Alkyl substituted triazoliums including Me, n-hexyl, and trifluoroethyl are also contemplated herein.

Accordingly, embodiments herein provide compounds of formulas (II), (III), (IV), (V), (VI), (VII), (VIII), (IX), (X), (XI), (XII), (XIII), and (XIV):

TABLE 1
Exemplary Substituted Phenyl (Ar) Groups
(I)

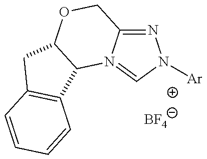

| | |
|---|---|
| 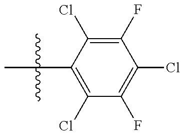 [SMILES: *c1c(Cl)c(F)c(Cl)c(F)c1Cl] | (II) |
| 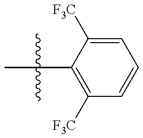 [SMILES: *c1c(C(F)(F)F)cccc1C(F)(F)F] | (III) |
| 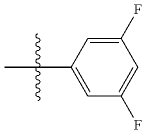 [SMILES: *c1cc(F)cc(F)c1] | (IV) |
| 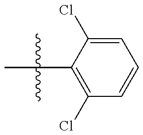 [SMILES: *c1c(Cl)cccc1Cl] | (V) |
| 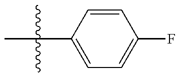 [SMILES: *c1ccc(F)cc1] | (VI) |
| 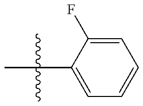 [SMILES: *c1ccccc1F] | (VII) |
| 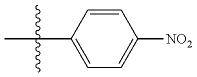 [SMILES: *c1ccc([N+](=O)[O-])cc1] | (VIII) |
| 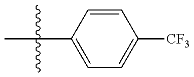 [SMILES: *c1ccc(C(F)(F)F)cc1] | (IX) |
| 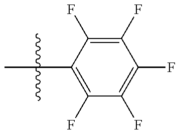 [SMILES: *c1c(F)c(F)c(F)c(F)c1F] | (X) |
| 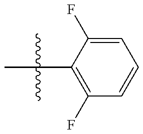 [SMILES: *c1c(F)cccc1F] | (XI) |
| 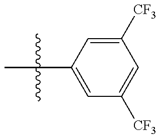 [SMILES: *c1cc(C(F)(F)F)cc(C(F)(F)F)c1] | (XII) |
| 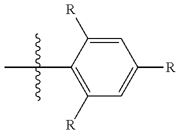 | (XIII) and (XIV) |

R = Me, Cl, or the combination thereof

Further illustrative embodiments provide compounds of formulas (XV), (XVI), (XVII), (XVIII), and (XIX):

TABLE 2
Additional Alternative Illustrative Ar Groups (1-napthyl, 2-napthyl,
2-pyridyl, 3-pyridyl, 4-pyridyl, 2-pyrimidinyl, 4-pyrimidinyl,
5-pyrimidinyl, 2-furanyl, 3-furanyl, 2-thiophene, 3-thiophene):

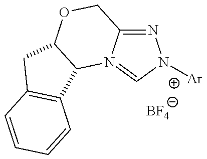

| Generic Ar Group | Potential Substituent Groups |
|---|---|
| 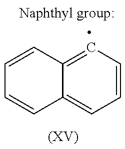 | X, RXₙ, RO, and NO₂, wherein R can be a substituted or unsubstituted branched or straight chain alkyl, X can be a halogen or pseudohalogen, and n is 1-3 |
| 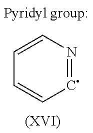 | X, RXₙ, RO, and NO₂, wherein R can be a substituted or unsubstituted branched or straight chain, alkyl, X can be a halogen or pseudohalogen, and n is 1-3 |
| 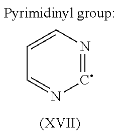 | X, RXₙ, RO, and NO₂, wherein R can be a substituted or unsubstituted branched or straight chain alkyl, Xcan be a halogen or pseudohalogen, and n is 1-3 |
| 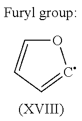 | X, RXₙ, RO, and NO₂, wherein R can be a substituted or unsubstituted branched or straight chain alkyl, X can be a halogen or pseudohalogen, and n is 1-3 |
| 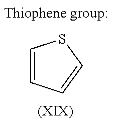 | X, RXₙ, RO, and NO₂, wherein R can be a substituted or unsubstituted branched or straight chain alkyl, X can be a halogen or pseudohalogen, and n is 1-3 |

Typical counter-ions for use with the compounds of formulas (I)-(XIX) include tetrafluoroborate (⁻BF₄), although other like charged molecules can also be used, including, for example, Cl, PF₆, BPh₄, and RBF₃.

The catalysts described herein are suitably loaded to reactions of the present invention as a mole percent of the reaction. In some embodiments the catalyst is loaded at 1 to 100 mole percent of the reaction, in other embodiments the catalyst is loaded at 10 to 50 mole percent of the reaction, and in some cases 10, 20, 30 or 40 mole percent of the reaction. In other embodiments, the reaction is performed super stoichiometrically—more than 100%.

In some embodiments, a combination of catalysts can be used in a particular reaction.

Finally, such catalysts provide unexpected and surprisingly robust control over the stereoselectivity of the products generated by the reactions described herein. Selection of a catalyst of a particular chirality will determine the particular enantiomeric form of the product. As shown in several reactions in the Examples, the chirality of the catalyst will determine the stereoselectivity of the H, F, Cl, or D introduced into the product. Illustratively, a catalyst of one configuration, such as that derived from (1R,2S)-(+)-cis-1-amino-2-indanol, can cause hydrogen introduction from the top to produce a stereocenter of R-configuration while a catalyst of opposite configuration, such as that derived from (1S,2R)-(−)-cis-1-amino-2-indanol, can cause hydrogen introduction from the bottom to produce a stereocenter of S-configuration.

### Synthesis of the Catalysts

The compounds of formulas (I)-(XIX) can be prepared by methods described herein. In a first embodiment, sodium hydride and n-pentane are combined under inert gas for 20 minutes. Sodium hydride is allowed to settle out of solution for 30 minutes and the n-pentane removed. Anhydrous tetrahydrofuran is added under inert gas and the solution placed in a 0 to 10° C. ice-brine bath with agitation for 30 minutes. (1R,2S)-(+)-cis-1-amino-2-indanol is added under inert gas with a second batch of (1R,2S)-(+)-cis-1-amino-2-indanol added. The reaction is allowed to proceed until evolution of hydrogen gas subsides, roughly 15 minutes. This reaction mix is heated in a 70° C. oil bath for 40 minutes with stirring under an inert gas atmosphere, followed by 1 hour in the 0-10° C. ice-brine bath. Ethyl chloroacetate is added over several minutes and the solution allowed to stir for approximately 30 minutes at room temperature, followed by 1 hour at 70° C. (under inert gas). The solution is then poured into a separation funnel and washed with brine. The combined aqueous layers are next back-extracted with ethyl acetate. The combined organics are stirred and MgSO₄ added; the solution is stirred for approximately 12 hours. The mixture is vacuum filtered to dryness and taken up in MTBE where it is agitated for 40 minutes. Vacuum filtration of the solid affords an off-white solid morpholinone (see synthesis schematic below, second structure).

In some embodiments, ethylene chloride and trimethyloxonium tetrafluoroborate are added to the morpholinone under inert gas and the mixture stirred at room temperature. Arylhydrazine is added and the mixture stirred. The solvent is removed in vacuo. Chlorobenzene and triethyl orthoformate or trimethyl orthoformate are added to the solution and heated to 100-130° C. with stirring for 24 hours, with the mixture open to the atmosphere.

In other embodiments, a catalyst may require that the hydrazide (see synthesis schematic below, third structure) be isolated before cyclization with orthoformate.

In still other embodiments, the above steps are rearranged to suit the particular synthesis reaction.

Use of other counterions can require a counterion exchange step.

The following is an exemplary catalyst synthesis reaction followed by a more general catalyst synthesis reaction (X represents any suitable counterion; R is aliphatic or aromatic):

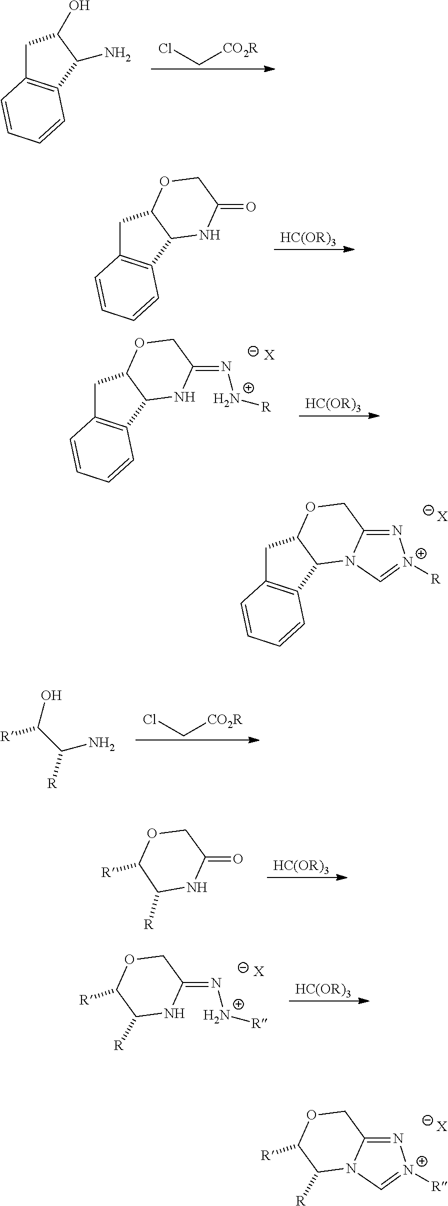

### Reactions Involving the Catalysts

Provided herein are methods for asymmetric hydration, fluorination, chlorination, and deuteration of α-reducible aldehydes such as enals. The method comprises contacting a suitable substrate, an enal, for example, with a compound of formula (I). In some embodiments, a method for asymmetric hydration comprises contacting the α-reducible aldehyde with a proton donor and a compound of formula (I) forming the respective carboxylic acid of the α-reducible aldehyde. In some embodiments, a method for asymmetric deuteration comprises contacting the α-reducible aldehyde with D₂O and a compound of formula (I), wherein the α-reducible aldehyde incorporates an α-deuteron. In other embodiments, a method for asymmetric fluorination comprises contacting the enal with a fluorine source and a compound of formula (I), wherein the enal incorporates the fluorine. Fluorine source herein refers to any of NFOBS, NFSim, Selectfluor, etc.

Suitable substrates include activated aldehydes or α-reducible aldehydes. Exemplary activated aldehydes included enals or any aldehyde that has a conjugated α-, β-unsubstituted or equivalent bond.

Each of the above asymmetric methods can be performed with a component of formula (I) or more particularly with a compound of formula (II)-(XIX).

Also provided are methods for asymmetric hydration, fluorination, chlorination, and deuteration of drug analogs. The method comprises contacting a drug analog with a compound of formula (I). In some embodiments, a method for asymmetric hydration comprises contacting the drug analog with a proton donor and a compound of formula (I). The drug analog can include at least one target aliphatic or functional group for asymmetric hydration to form the acid of the drug analog.

The reactions herein are amenable to a variety of substitution on the backbone and reducible aldehydes. R can be alkyl, cycloalkyl, aryl, and heteroaryl. In the case of enals, both alkene geometries may be used as well as terminal alkenes (R equals H) and tetrasubstituted alkenes bearing carbon and heteroatom substitution. See also Reaction Schemes 1 and 2.

The following catalysts are a representative but not exclusive subset of potential catalysts for the reaction: triazolium, thiazolium and imidazolium catalysts with or without fused rings bearing alkyl, aryl and heteroaryl substitution about the core as well as stereocenters in various positions.

In general, hydration reactions catalyzed using the compounds disclosed herein can be performed as follows: various catalysts can conduct the reaction on α-reducible aldehydes such as enals, halo-aldehydes, epoxy-aldehydes, aziridino aldehydes, and cyclopropane carboxaldehydes, in the presence of a variety of bases under aqueous conditions or mixed solvent conditions (organic solvent in the presence of water), or in water. Alternatively, a nucleophile such as one or more alcohols can be substituted for the water (forming the respective ester rather than the carboxylic acid).

In general, halogenation reactions, e.g. fluorination reactions or chlorination reactions, catalyzed using the compounds disclosed herein can be performed as follows: various catalysts may conduct the reaction on α-reducible aldehydes such as enals, halo-aldehydes, epoxy-aldehydes, aziridino aldehydes, and cyclopropane carboxaldehydes. The reaction is performed in the presence of a variety of bases under aqueous conditions or mixed solvent conditions (organic solvent in the presence of water) or in water, or performed under conditions that do not contain appreciable amounts of water, in the presence of an electrophilic fluorine source (for fluorination) such as NFSI among others, or in the presence of an electrophilic chlorine source (for chlorination) such as NCS among others.

In general, deuteration reactions catalyzed using the compounds disclosed herein can be performed as follows: various catalysts may conduct the reaction on α-reducible aldehydes such as enals, halo-aldehydes, epoxy-aldehydes, aziridino aldehydes, and cyclopropane carboxaldehydes, in the presence of a variety of bases under aqueous conditions or mixed solvent conditions (organic solvent in the presence of water) or in water, wherein the water is heavy water (or D₂O), or alternately in deuterated alcohol, the latter delivering the ester instead of the carboxylic acid.

The reactions can occur at temperatures as low as about −40° C. or as high as about 110° C. In some embodiments, the reactions are fast and can be as short as minutes. In other embodiments, the reactions can take hours to several days to generate product.

The reaction can be performed at various scales, for example, from mg to grams or on a very large scale for industrial purposes or pharmaceutical manufacturing purposes.

The reaction is well suited to be conducted in non-polar solvents such as toluene, hexanes, benzene, and pentanes, but works in other solvents as well, such as ethereal solvents (including diethyl ether, tetrahydrofuran), halogenated solvents (including dichloromethane), or alcoholic solvents such as tert-butanol. One can expect some degree of reaction in many, many different solvents including solventless (neat) or under aqueous conditions alone (in water or on water).

A variety of inorganic bases or organic bases also facilitate the reaction such as but not limited to K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaHCO₃, KHCO₃, CsHCO₃, K₂HPO₄, KH₂PO₄, KOAc, and NaOAc.

Potential phase transfer reagents include, but are not limited to TBACl, TBAI, and TBAOH, as well as other similar agents. Exemplary dehydrating solutions include aqueous saturated NaCl, saturated NaBr, and saturated NaI.

Some embodiments herein, particularly hydration reactions, can also include brine or other water saturated with a halogen solution, e.g., water saturated with sodium bromide, water saturated with sodium iodine, etc. In some embodiments, a silanol can be used as the nucleophile delivering the carboxylic acid on work-up.

As for equivalents relative to substrate: of water, from one to very large excess, base from less than 1 equivalent to much more than one equivalent (10 or greater), concentration in solvent from very dilute (0.001 M) to solventless (very concentrated).

As used herein, the term “additive” refers to any one of or combination of (a) phase transfer reagents including, but not limited to tetraalkyl ammonium salts such as TBACl, TBAI, and TBAOH, and/or (b) brine, including, but not limited to water supersaturated with sodium chloride, water saturated with sodium bromide, or water saturated with sodium iodide.

In some embodiments, any amount of additive can be contacted with the catalyst to provide enhanced product formation. In some aspects, a 1:1 molar ratio of additive to catalyst provides an unexpectedly high yield of the desired enantiomer. See, for example, Examples 6 and 7. In other aspects, the additive is present in an excess amount relative to the amount of catalyst present in the reaction.

As used herein, the phrase enantiomeric excess refers to the absolute difference between the mole fraction of each enantiomer.

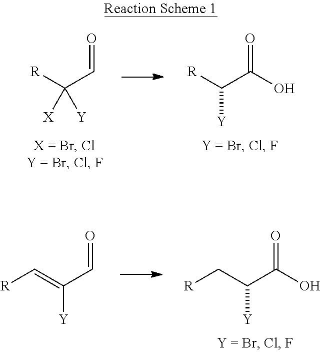

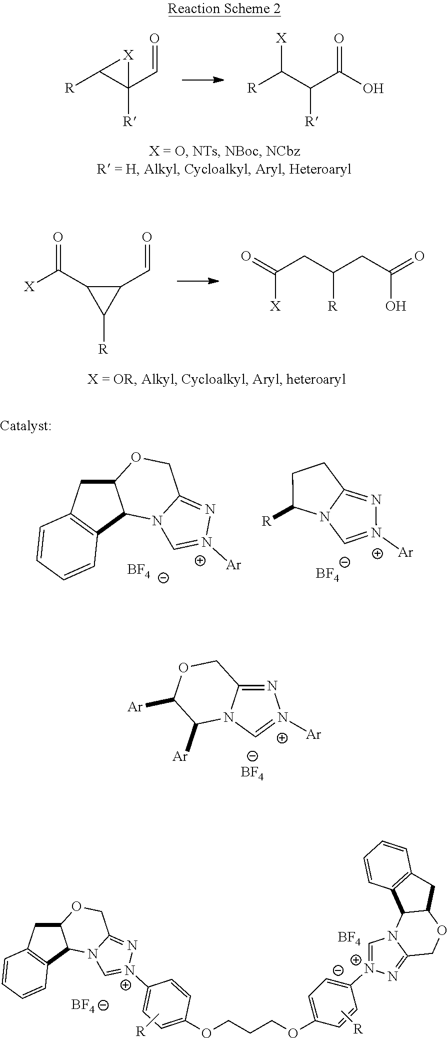

When used herein, the term “halogen atom” or “halo” include fluorine, chlorine, bromine and iodine and fluoro, chloro, bromo, and iodo, respectively.

When used herein, the term “alkyl” includes all straight and branched isomers. Representative examples of these types of groups include methyl, ethyl, propyl, isopropyl, butyl, isobutyl, tert-butyl, sec-butyl, pentyl, hexyl, heptyl, and octyl.

When used herein, the term “cycloalkyl” includes cyclic isomers of the above-described alkyls. Exemplary cycloalkyls include cyclopropyl, cyclobutyl, cyclopentyl, cyclohexyl, cycloheptyl, and cyclooctyl.

“Aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Exemplary aryl groups contain one aromatic ring or 2 to 4 fused or linked aromatic rings, e.g., phenyl, naphthyl, biphenyl, and the like. “Substituted aryl” refers to an aryl moiety substituted with one or more substituent groups, and the terms “heteroatom-containing aryl” and “heteroaryl” refer to aryl in which at least one carbon atom is replaced with a heteroatom. Typically the heteroaryl will contain 1-2 heteroatoms and 3-19 carbon atoms. Unless otherwise indicated, the terms “aryl” and “aromatic” includes heteroaromatic, substituted aromatic, and substituted heteroaromatic species. Illustrative aryls include phenyl, naphthyl, benxyl, tolyl, xylyl, thiophene, indolyl, etc. Illustrative heteroaryls include substituted or unsubstituted furyl, thiophenyl, pyridyl, pyrimidyl, and other heteroatom containing aromatics.

When used herein, the term “substituted” means that one or more hydrogens on the designated atom is replaced with a selection from the indicated group.

As used herein, the phrase “having the formula” or “having the structure” is not intended to be limiting and is used in the same way that the term “comprising” is commonly used.

“Stereoselective” refers to a chemical reaction that preferentially results in one stereoisomer relative to a second stereoisomer, i.e., gives rise to a product in which the ratio of a desired stereoisomer to a less desired stereoisomer is greater than 1:1.

### EXAMPLES

The following examples are provided for illustrative purposes only and are not intended to limit the scope of the invention. The chart below provides abbreviations and acronyms used herein with the full word or phrase spelled out.

| | |
|---|---|
| MTBE | Methyl tertiary butyl ether |
| DCM | Dichloromethane |
| TBAI | Tetrabutylammonium |
| | iodide |
| NCS | N-Chlorosuccinimide |
| THF | Tetrahydrofuran |
| brine | water supersaturated with |
| | sodium chloride, water |
| | saturated with sodium |
| | bromide, or water saturated |
| | with sodium iodide |

Carboxylic acid products were converted to their methyl ester derivative for ease of structural characterization by LCMS or GCMS. Unless described otherwise a portion of the acid product is dissolved in 1:1 DCM and MeOH and to the vessel was added CH₂N₂ dropwise with stirring until bubbling subsided and the solution turned yellow. Excess diazomethane was quenched via the addition of AcOH. The reaction was concentrated in vaccuo to yield the methyl ester.

### Example 1

### Catalyst Synthesis, Part 1

A flame dried 2.0 L, single neck, round-bottomed flask fitted with magnetic stirrer, argon inlet and a rubber septum was charged with sodium hydride (2.73 g, 60% in mineral oil, 68.28 mmol, 1.02 equiv) via a powder funnel, followed by n-pentane (300 mL). The septum was then replaced and the heterogenous mixture was subjected to an argon atmosphere followed by agitation for 20 minutes. Agitation was stopped and the sodium hydride was allowed to settle out of solution for 30 minutes. n-Pentane was then removed via cannula. The septum was briefly removed to add anhydrous tetrahydrofuran (1.0 L) via powder funnel followed by replacement of the septum and resubjection to an argon atmosphere. The solution was placed in a 0 to −10° C. ice-brine bath with agitation and allowed to cool for 30 minutes. The septum was then replaced with a powder funnel and (1R,2S)-(+)-cis-1-amino-2-indanol (5 g, 33.52 mmol) was added followed by replacement of the septum and resubjection to an argon atmosphere. Color change was observed to a light purple heterogeneous solution. This procedure was then repeated with an additional 5 g batch of (1R,2S)-(+)-cis-1-amino-2-indanol and agitated until evolution of hydrogen gas had subsided (approx. 15 minutes). The flask was then fitted with a reflux condenser and heated in a 70° C. oil bath for 40 minutes with stirring under an argon atmosphere. The solution was then placed in a 0 to −10° C. ice-brine bath and allowed to cool for 1 hour while stirring. Ethyl chloroacetate (8.38 g, 7.32 mL, 68.37 mmol, 1.02 equiv) was added via syringe over 5 minutes. The solution was removed from the ice-brine bath and stirred for 30 minutes at room temperature. The solution was then placed in 70° C. oil bath and stirred for 1 hour under argon. After cooling to room temperature, the homogeneous deep purple solution was poured into a 2.0 L separation funnel and washed with brine (2×200 mL). The combined aqueous layers were then back extracted with ethyl acetate (2×200 mL). The combined organics were then poured into a 2.0 L round bottom flask with magnetic stir bar and stirred vigorously. To the flask was added anhydrous MgSO₄ (50 g) via powder funnel and stirring was continued for 12 hours. The mixture was then vacuum filtered through a coarse fritted funnel into a 1.0 L round bottom flask in approx. 500 mL portions and concentrated in vacuo to dryness. To the 1.0 L round bottom flask containing the crude light brown solid was added a stir bar and hexanes (300 mL). The sides of the round bottom flask were scraped with a metal spatula to ensure that all crude solid material rested in the bottom of the flask. The flask was fitted with a reflux condenser left open to atmosphere and the heterogeneous mixture was stirred vigorously in a 70° C. oil bath for a period of 2 hours. After cooling to room temperature, vacuum filtration afforded an off-white solid which was placed in a 100 mL round bottom flask via powder funnel and dried under vacuum (2 mmHg) in a 70° C. oil bath for 1 hour, affording 10.64-11.15 g (84-88%) of morpholinone (see synthesis schematic above).

### Example 1

### Catalyst Synthesis, Part 2

To a flame-dried 1.0 L round bottom flask with magnetic stir bar was added morpholinone (10.00 g, 52.85 mmol, 1.0 equiv) via powder funnel. The flask was then evacuated and back-filled with argon. Methylene chloride (300 mL) and trimethyloxonium tetrafluoroborate (7.82 g, 52.85 mmol, 1.0 equiv) were then added via powder funnel and the flask was fitted with a septum followed by subjection to an argon atmosphere. The heterogeneous mixture was stirred at room temperature until the reaction was homogeneous. Pentafluorophenylhydrazine (10.47 g, 52.85 mmol, 1.0 equiv) was added in a single portion via powder funnel followed by replacement of the septum and stirred for 4 hours. The magnetic stir bar was removed followed by removal of the solvent in vacuo. The 1.0 L flask was then placed in a 100° C. oil bath for 1 hour under full vacuum (2 mmHg). A magnetic stirring bar, chlorobenzene (300 mL) and triethyl orthoformate (19.58 g, 21.97 mL, 132.13 mmol, 2.5 equiv) were then added, the flask was fitted with a reflux condenser and placed in a 130° C. oil bath and stirred for 24 hours open to the atmosphere. Triethyl orthoformate (19.58 g, 21.97 mL, 132.13 mmol, 2.5 equiv) was then added via syringe followed by continued agitation for 24 hours. A third portion of triethyl orthoformate (19.58 g, 21.97 mL, 132.13 mmol, 2.5 equiv) was added via syringe followed by continued agitation for 24 hours. After removal of the reaction vessel from the oil bath, and cooling to room temperature, the solution was added to a 1.0 L round bottom flask containing toluene (300 mL) that was then agitated with a magnetic stir bar. The reaction vessel was then rinsed with toluene (50 mL) followed by addition of the heterogeneous mixture to the 1 L flask containing the crude product. The slurry was stirred for 10 minutes followed by vacuum filtration. The filtrate was rinsed with toluene (200 mL) and hexane (200 mL). The solid was then transferred to a 125 ml Erlenmeyer flask containing a stir bar by powder funnel and triturated with 20 ml of ethyl acetate and 5 ml of methanol and stirred vigorously for 30 min. The slurry was then filtered and the filter cake is washed with 15 ml of cold ethyl acetate via glass pipette to yield azolium salt as an off white solid The off white solid was transferred to a 100 mL round bottom flask via powder funnel and placed in a 100° C. oil bath and subjected to vacuum (2 mmHg) for 1 hour, affording 13.57-16.80 g (55-68%).

Exemplary catalysts synthesized according to the method described above are shown below:

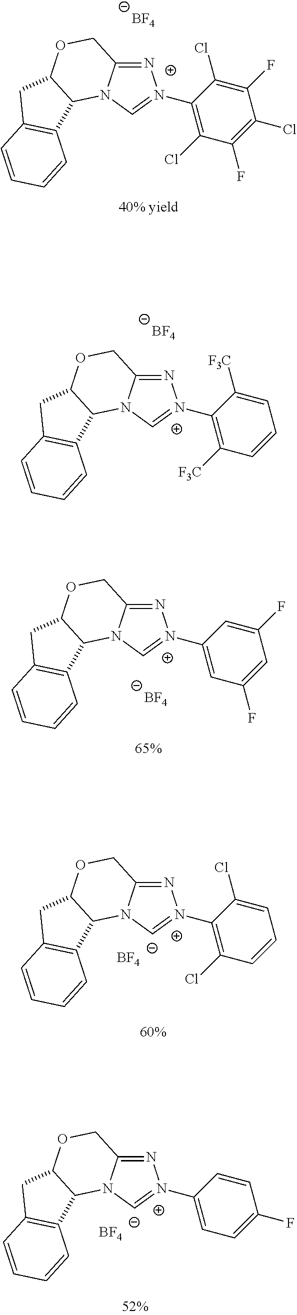

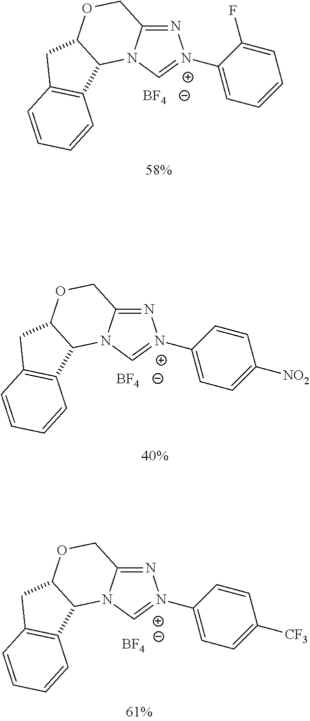

### Example 2

### Diazonium Tetrafluoroborate Salt Synthesis

250 ml of anhydrous DCM was added to a flame dried 500 ml three neck round bottom flask. Boron trifluoride diethyl etherate (16.48 g, 116.18 mmol, 1.5 equiv) was added in one portion at −15° C. Aniline (10.00, 77.45 mmol, 1.0 equiv) was then added dropwise at −15° C. to generate a white precipitate. Anhydrous ether was added until a homogeneous solution was observed. t-butyl nitrite (9.58 g, 92.94 mmol, 1.2 equiv) was then added dropwise at −15° C., until a precipitate was observed. The reaction vessel was warmed to 0° C. and 100 ml of pentanes was added and stirred at 25° C. The solid is then filtered and dried to afford the product in 72% yield.

### Example 3

### Difluoro Hydrazine Synthesis

A 250 mol round bottom flask was charged with diazonium tetrafluoroborate salt (1.0 g, 1.0 equiv) dissolved in 100 ml of HCl and cooled to −40° C. To the agitated homogeneous solution was added a solution of SnCl₂ dihydrate (XX, XX, 2.0 equiv) in HCl. The mixture was stirred for 2 hours at −40° C. and then neutralized to pH 7 upon slow addition of K₂CO₃. The reaction was extracted with ethyl acetate and washed with brine to furnish the crude hydrazine. The hydrazine was used without further purification.

### Example 4

### α-Bromo Carboxylic Acid Synthesis

To a flame dried 25 ml round bottom flask with magnetic stir bar was charged triazolium salt catalyst IV (0.017, 0.041 mmol, 20 mol %) and TBAI (0.008, 0.021 mmol, 10 mol %). Aldehyde was added to the flask (0.060, 0.205 mmol, 1.0 eq) followed by toluene (0.02M with respect to aldehyde). The flask was purged with argon and brine was added to the reaction vessel (of equal volume to 1.0 eq of 1M K₂CO₃ in H₂O). The reaction was stirred under an atmosphere of argon for 5 minutes followed by addition of 1.0 equiv of 1M K₂CO₃ in H₂O. The reaction was stirred vigorously until completion (12-28 hours) followed by addition of 3.0 equiv of AcOH to the reaction vessel. The reaction mixture was loaded on to a plug of silica and eluted with EtOAc with 5% AcOH. The crude solution was reduced in vaccuo to yield an oil. The crude oil was taken up in toluene and the solution reduced in vaccuo. The crude oil was taken up in toluene and purified via column chromatography via gradient elution to produce the desired acid product after evaporation of chromotagraphy solvent in vacuo.

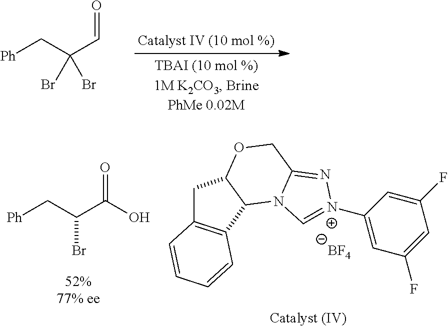

### Example 5

### α-Bromo, α-Deuterio Carboxylic Acid Synthesis

To a flame dried 25 ml round bottom flask with magnetic stir bar was charged triazolium salt catalyst IV (0.017, 0.041 mmol, 20 mol %) and TBAI (0.008, 0.021 mmol, 10 mol %). To the flask was added aldehyde (0.060, 0.205 mmol, 1.0 eq) followed by toluene (0.02M with respect to aldehyde). The flask was purged with argon and brine (NaCl in D₂O) was added to the reaction vessel (of equal volume to 1.0 eq of 1M K₂CO₃ in D₂O). The reaction was stirred under an atmosphere of argon for 5 minutes followed by addition of 1.0 equiv of 1M K₂CO₃ in D₂O. The reaction was stirred vigorously until completion (12-28 hours) followed by addition of 3.0 equiv of AcOH to the reaction vessel. The reaction mixture was loaded on to a plug of silica and eluted with EtOAc with 5% AcOH. The crude solution was reduced in vaccuo to yield an oil. The crude oil was taken up in toluene and the solution reduced in vaccuo. The crude oil was taken up in toluene and purified via column chromatography via gradient elution to produce the desired acid product after evaporation of chromotagraphy solvent in vacuo.

### Example 6

### β-Hydroxy, α-Methyl Carboxylic Acid Synthesis

To a flame dried 25 ml round bottom flask with magnetic stir bar was charged triazolium salt catalyst B (0.026, 0.074 mmol, 0.02 equiv), and TBAI (0.014, 0.037, 0.01 equiv). To the flask was added aldehyde (0.060, 0.370 mmol, 1.0 equiv) followed by toluene (0.02 m with respect to aldehyde). The flask was purged with argon and brine was added to the reaction vessel (of equal volume to 1.0 eq of 1M K₂CO₃ in H₂O). The reaction is stirred under an atmosphere of argon for 5 minutes followed by addition of 1.0 equiv of 1M K₂CO₃ in H₂O. The reaction was stirred vigorously until completion (12-28 hours) followed by addition of 3.0 equiv of AcOH to the reaction vessel. The reaction mixture was loaded on to a plug of silica and eluted with EtOAc with 5% AcOH. The crude solution was reduced in vaccuo to yield an oil. The crude oil was taken up in toluene and the solution reduced in vaccuo. The crude oil was taken up in toluene and purified via column chromatography via gradient elution to produce the desired acid product after evaporation of chromotagraphy solvent in vacuo.

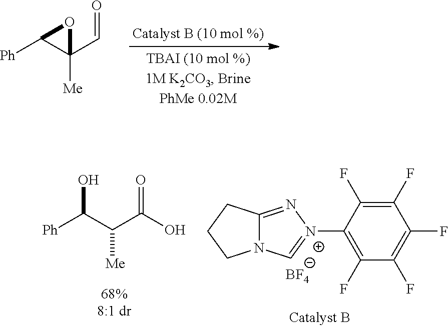

### Example 7

### β-Amino, α-Methyl Carboxylic Acid Synthesis

To a flame dried 25 ml round bottom flask with magnetic stir bar was charged triazolium salt catalyst B (0.026, 0.074 mmol, 0.02 equiv), and TBAI (0.014, 0.037, 0.01 equiv). To the flask was added aldehyde (0.060, 0.370 mmol, 1.0 equiv) followed by toluene (0.02 m with respect to aldehyde). The flask was purged with argon and brine was added to the reaction vessel (of equal volume to 1.0 eq of 1M K₂CO₃ in H₂O). The reaction was stirred under an atmosphere of argon for 5 minutes followed by addition of 1.0 equiv of 1M K₂CO₃ in H₂O. The reaction was stirred vigorously until completion (12-28 hours) followed by addition of 3.0 equiv of AcOH to the reaction vessel. The reaction mixture was loaded on to a plug of silica and eluted with EtOAc with 5% AcOH. The crude solution was reduced in vaccuo to yield an oil. The crude oil was taken up in toluene and the solution reduced in vaccuo. The crude oil was taken up in toluene and purified via column chromatography via gradient elution to produce the desired acid product after evaporation of chromotagraphy solvent in vacuo.

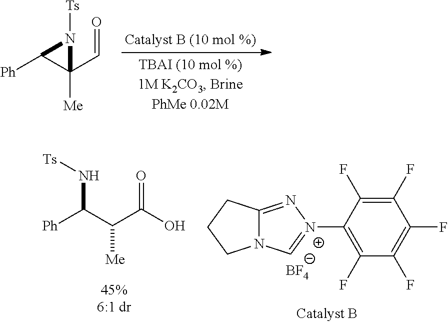

### Example 8

### Two Step One Pot Hydration

To a flame dried 25 ml round bottom flask with magnetic stir bar was charged rac-proline (0.005, 0.045 mmol, 0.1 equiv) followed by 5 ml of anhydrous DCM. The reaction vessel was cooled to 0° C. followed by addition of aldehyde (0.060, 0.447 mmol, 1.0 equiv) and NCS (0.060, 0.447 mmol, 1.0 equiv). The reaction was stirred at 0° C. for 1 hour and warmed to room temperature and stirred for an additional 2 hours. The reaction vessel was then charged with azolium salt B followed by 5 ml of DCM. To the solution at 25° C. was added brine (of equal volume to 1.0 eq of 1M K₂CO₃ in H₂O). The reaction was stirred under an atmosphere of argon for 5 minutes followed by addition of 2.5 equiv of 1M K₂CO₃ in H₂O. The reaction was agitated for 24 hours followed by 5 equiv of AcOH. The crude solution was reduced in vaccuo to yield an oil. The crude oil was taken up in toluene and the solution reduced in vaccuo. The crude oil was taken up in toluene and purified via column chromatography via gradient elution to produce the desired acid product after evaporation of chromotagraphy solvent in vacuo.

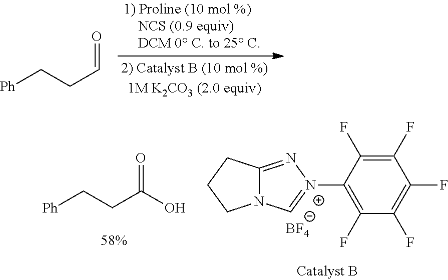

### Example 9

### Miscellaneous Related Reactions

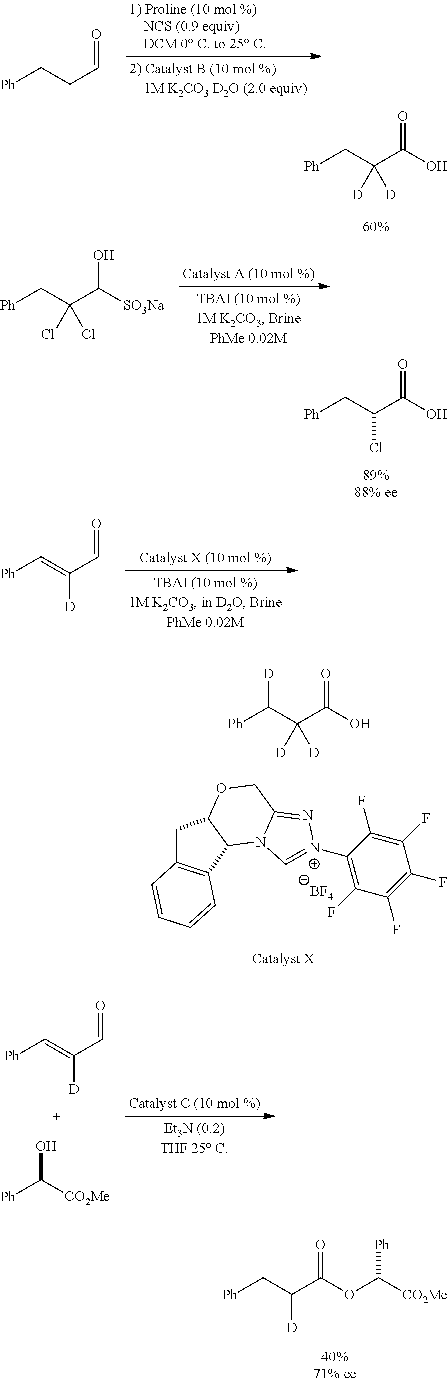

### Example 10

### Mechanism of NHC Redox Process

α,α-dichloro aldehydes in the presence of a chiral N-heterocyclic carbene (NHC) and phenol can yield the respective α-chloro aryl ester in good yields and ee's. In an effort to maximize both atom- and step-economy in these processes, water was used as the nucleophile to generate the α-halo acids directly, the redox hydration of the α-reducible aldehyde. The result is the asymmetric synthesis of α-chloro and α-fluoro carboxylic acids through a mild biphasic redox process. The versatility of the developed reaction also lends itself towards the incorporation of a deuterium by simply using D₂O.

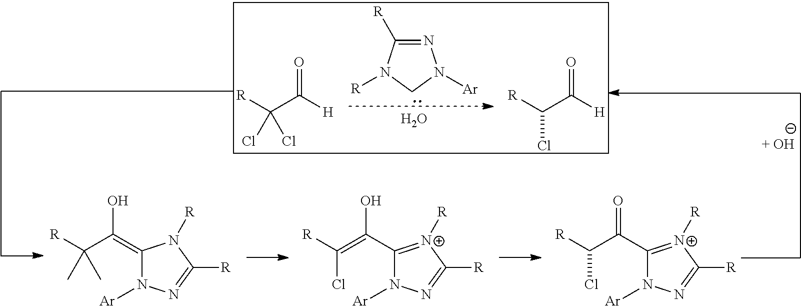

### Example 11

### Synthesis of α-Chloro Carboxylic Acids

Carbonate bases were identified as being optimal in an initial screen. Investigations into the catalyst revealed that catalyst (X), previously demonstrated as the optimal precatalyst in our asymmetric synthesis of α-chloro esters, affords the α-chloro acid in 89% yield and 78% ee (eq 1). Efforts to determine the effect of the carbene on the reaction showed that a sufficiently electron withdrawing 2,6-difluorophenyl group catalyst (XI) was necessary to facilitate reactivity (87% yield) with an increase in the enantioselectivity to 87%. Electron deficient 3,5-bis(trifluoromethyl)phenyl catalyst (XII), and sterically hindered mesityl derivative catalyst (XIII) lead to no reaction. In order to obtain the reactivity observed with catalyst (X) while mimicking the sterics of precatalyst (XIII), catalyst (XIV) was synthesized which generates the desired product in 91% yield and 77% ee.

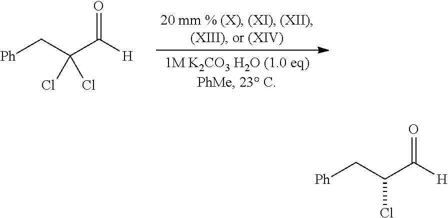 [SMILES: O=CC(Cl)(Cl)Cc1ccccc1 ; O=C[C@H](Cl)Cc1ccccc1]

The use of alcohols instead of water delivers the corresponding ester instead of carboxylic acid.

### Example 12

### Catalyst Stoichiometry and Additive Effect

Increasing the stoichiometric ratio of catalyst to substrate improved enantioselectivities and yield up to a threshold of no further improvement (Entries 1-4, Table 3). Importantly the use of phase transfer agents such as TBAI, as additives, improved both the enantioselectivity and yields over that of the same mol % catalyst without the additives (entry 1 vs. entry 6 or 7, in Table 3).

TABLE 3
Effect of Catalyst (XI) on Selectivity and Reactivity.

| entry^{a} | mol % | additive | yield (%) | ee (%)^{b} |
|---|---|---|---|---|
| 1 | 10 | — | 50 | 80 |
| 2 | 20 | — | 89 | 87 |
| 3 | 30 | — | 90 | 90 |
| 4 | 40 | — | 90 | 92 |
| 5 | 10 | brine | 60 | 87 |
| 6 | 10 | Bu₄NI | 85 | 87 |
| 7 | 10 | brine/ | 89 | 88 |
| | | Bu₄NI | | |

^{a}All reactions conducted in PhMe (0.02M) at 23° C.
^{b}Ee's determined on the derived methyl ester by HPLC analysis on a chiral stationary phase.

### Example 13

### Hemiaminal Formation

Several modes by which the carbene may interact with water (Scheme 3) are contemplated. Protonation of the carbene with water may lead to azolium hydroxide, which can act as a phase-transfer agent supplying hydroxide into the organic phase. Additionally, diastereomeric hemiaminals may form the hydrate of carbene. Residual water in the organic phase may serve as the proton source as shown in Example 11 (water was used as the nucleophile to generate the α-halo acids directly), thus brine was introduced as an additive and to increase both yield and ee (entry 5, Table 3). In an attempt to probe the role of the azolium as phase transfer agent, 10 mol % tetrabutylammonium iodide (Bu₄NI) was added resulting in improved reactivity and selectivity (entry 6, Table 3). The use of both brine and Bu₄NI proved to be optimal (entry 7, Table 3).

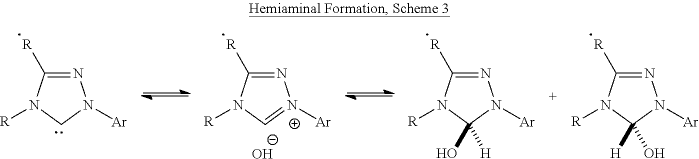

### Example 14

### Formation of α-Chloro Carboxylic Acids

A variety of α-dichloro aldehydes participate in this reaction. Substitution of D₂O in place of H₂O leads to an asymmetric deuteration reaction affording enantioenriched isotopically labeled chloroacids, of potential interest as drug analogs. Ortho-methoxy and para-N-Bocamino groups are each tolerated on the aromatic ring B and C yielding the respective products in 78-80% and 78-95% ee. It is interesting to note that presence of an additional proton donor in C significantly reduces the ee presumably due to competitive protonation. Analogs bearing aliphatic groups D and E and functional groups F, G, and H all yield the acid in good yield (75-86%) and 89-91% ee. See (Table 4).

TABLE 4
Scope of α chloro-carboxylic acids.

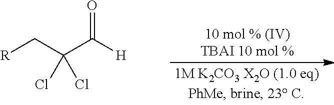

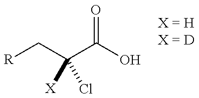

| | |
|---|---|
| 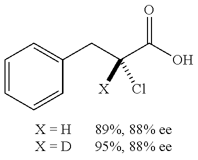 | A |
| 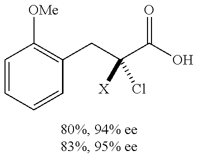 | B |
| 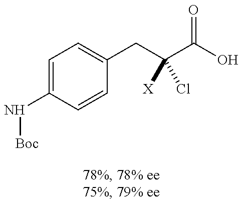 | C |
| 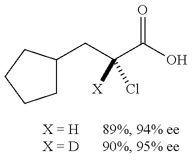 | D |
| 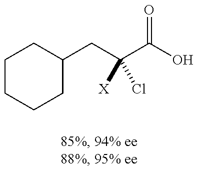 | E |
| 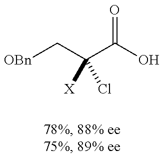 | F |
| 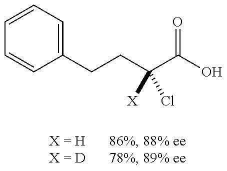 | G |
| 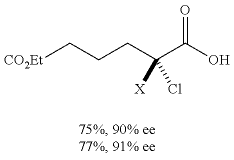 | H |

All reactions conducted in PhMe (0.02M) at 23° C.
Ee's determined on the derived methyl ester by HPLC analysis on a chiral stationary phase.

### Example 15

### Formation of α-Fluoro Carboxylic Acids

α-Fluoro carboxylic acids are attractive products for the pharmaceutical industry as fluorine is an isostere for hydrogen. α-Fluoroenals were chosen as the redox partner for this mild catalytic process, and a mixture of olefin isomers were tolerated. Several points are worthy of note: the use of TBAI leads to enal decomposition and higher yields were observed using KHCO₃ in place of K₂CO₃. Subjection of the aldehyde to the optimized conditions yields the respective α-fluoro carboxylic acids in excellent yields and enantioselectivities. Aromatic and heteroaromatic I-L (Entries 1-4, Table 5) fluoroenals yield the α-fluoro carboxylic acids in 70-80% yield and 90-96% ee. Aliphatic enals are also suitable substrates with M formed in 65% yield and 96% ee (Entry 5, Table 5).

TABLE 5
Scope of α Fluoro-Carboxylic Acids

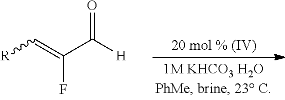

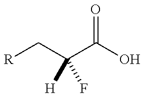

| | |
|---|---|
| 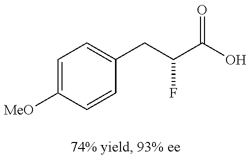 | I |
| 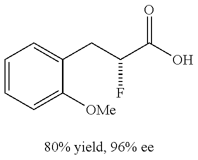 | J |
| 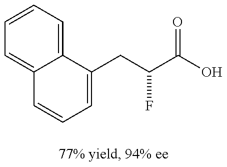 | K |
| 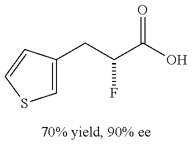 | L |
| 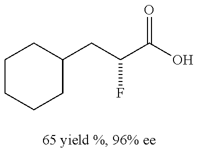 | M |

a All reactions conducted in PhMe (0.02M) at 23° C.
b Ee's determined on the derived methyl ester by HPLC analysis on a chiral stationary phase.

### Example 16

### Formation of α-Deutero Carboxylic Acids

The above described experiments show that enantioenriched α-chloro and α-fluoro carboxylic acids can be accessed in a catalytic asymmetric manner. The versatility of the developed reaction also lends itself to a mild and inexpensive method for the incorporation of an α-deuteron in an asymmetric fashion using D₂O.

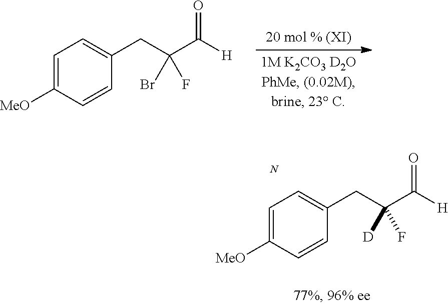

An exemplary enantioenriched α-deutero α-fluoro carboxylic acid was obtained by subjecting the α-bromo α-fluoro aldehyde shown in the above equation to the optimized conditions forming acid N in 77% yield and 96% ee.

### Example 17

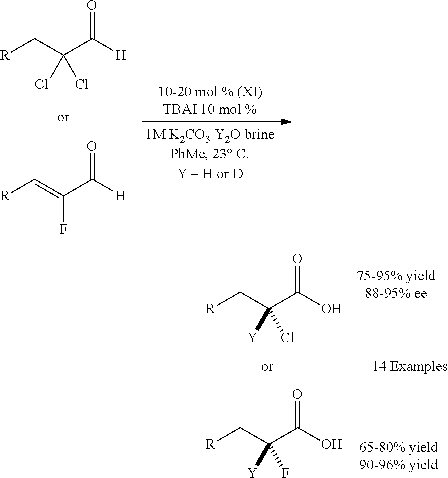

### Example 18

### General Methods

All reactions were carried out under an atmosphere of argon using flame-dried glassware with magnetic stirring. Toluene was degassed and passed through one column of neutral alumina and one column of Q5 reactant. Column chromatography was performed on SiliCycle® Silica Flash® 40-63 μm 60A. Thin Layer chromatography was performed on SiliCycle® 250 μm 60A plates. Visualization was accomplished with UV light, KMnO₄, bromocrescol Green stain followed by heating.

¹H NMR and ¹³C NMR spectra were recorded on Varian 400 MHz spectrometers at ambient temperature. ¹H NMR data were reported as follows: chemical shift in parts per million (6, ppm) from chloroform (CHCl₃) taken as 7.26 ppm, integration, multiplicity (s=singlet, d=doublet, t=triplet, q=quartet, m=multiplet) and coupling constant (Hz). ¹³C NMR was reported as follows: chemical shifts in ppm from CDCl₃ taken as 77.0 ppm. Mass spectra were obtained on a Fisons VG Autospec.

¹H NMR and ¹³C NMR spectra of the azolium salts were recorded on Varian 400 MHz spectrometers at ambient temperature. ¹H NMR data were reported as follows: chemical shift in parts per million (δ, ppm) from chloroform (CHCl₃) taken as 7.26 ppm, integration, multiplicity (s=singlet, d=doublet, t=triplet, q=quartet, m=multiplet) and coupling constant (Hz). ¹³C NMR were reported as follows: chemical shifts in ppm from CDCl₃ taken as 77.0 ppm.

α,α-dichloro aldehydes were prepared according to literature procedure from the corresponding aldehyde and freshly distilled prior to use or purified by generating the bisulfite adduct. Aldehydes A, E, and G from Example 14 match spectrometric data and physical properties to those previously reported in the literature. Bisulfite adducts were prepared according to literature procedure from the corresponding α,α-dichloro aldehydes and dried under vacuum prior to use. α-Fluoro enals were prepared according to literature procedure. Bisulfite adducts were prepared according to literature procedure from the corresponding α-Fluoro enals and dried under vacuum prior to use. α-bromo α-fluoro aldehyde was prepared according to literature procedure and distilled prior to use.

Distilled water was used without further purification. Deuterium Oxide was purchased from Cambridge Isotope Laboratories, Inc and was used without further purification. Anhydrous Potassium Carbonate was purchased from Fisher Scientific and used without further purification. Sodium Chloride was purchased from Fisher Scientific and used without further purification. Diazomethane was prepared according to literature procedure from Diazald.

All racemic products were obtained upon treating the respective aldehyde with achiral triazolium salt using DCM as solvent and 1M K₂CO₃.

### Example 19

### General Synthesis Procedures

General Procedure (A) for the Synthesis of α-Proteo, α-Chloro Carboxylic Acid:

To a flame dried 25 ml round bottom flask with magnetic stir bar was added triazolium salt XI (0.012, 0.029 mmol, 10 mol %) and TBAI (0.011, 0.029 mmol, 10 mol %). To the flask was added the α-chloro aldehyde of Example 11, 2-chloro-3-phenylpropanal, (0.060, 0.295 mmol, 1.0 eq) followed by toluene (0.02M with respect to aldehyde). The flask was purged with argon and brine was added to the reaction vessel (of equal volume to 1.0 eq of 1M K₂CO₃ in H₂O). The reaction was stirred under an atmosphere of argon for 5 minutes followed by addition of 1.0 equiv of 1M K₂CO₃ in H₂O. The reaction was stirred vigorously until completion (12-28 hours) followed by addition of 3.0 equiv of AcOH to the reaction vessel. The reaction mixture was loaded on a plug of silica and eluted with EtOAc with 5% AcOH to produce the desired acid product after evaporation of chromotagraphy solvent in vacuo.

General Procedure (B) for the Synthesis of α-Deutero, α-Chloro Carboxylic Acid:

To a flame dried 25 ml round bottom flask with magnetic stir bar was added triazolium salt XI (0.012 g, 0.0295 mmol, 10 mol %) and TBAI (0.011 g, 0.0295 mmol, 10 mol %). To the flask was added the α-chloro aldehyde of Example 11, 2-chloro-3-phenylpropanal, (0.060 g, 0.295 mmol, 1.0 eq) followed by toluene (0.02M with respect to aldehyde). The flask was purged with argon and brine (saturated solution of NaCl in D₂O) was added to the reaction vessel (of equal volume to 1.0 eq of 1M K₂CO₃ in D₂O). The reaction was stirred under an atmosphere of argon for 5 minutes followed by addition of 1.0 equiv of 1M K₂CO₃ in D₂O. The reaction was stirred until completion (8-10 hours) followed by addition of 1.0 equiv of AcOH to the reaction vessel. The reaction mixture was loaded on to a plug of silica and eluted with EtOAc with 5% AcOH to produce the desired acid product after evaporation of chromotagraphy solvent in vacuo.

General Procedure (C) for the Synthesis of α-Proteo, α-Fluoro Carboxylic Acid:

To a flame dried 25 ml round bottom flask with magnetic stir bar was added triazolium salt XI (0.028 g, 0.066 mmol, 20 mol %). To the flask was added an α-fluoro aldehyde (0.060 g, 0.333 mmol, 1.0 eq) followed by toluene (0.02M with respect to aldehyde). The flask was purged with argon and brine was added to the reaction vessel (of equal volume to 1.0 eq of 1M KHCO₃ in H₂O). The reaction was stirred under an atmosphere of argon for 5 minutes followed by addition of 1.0 equiv of 1M KHCO₃ in H₂O. The reaction was stirred until completion (8-10 hours) followed by addition of 1.0 equiv of AcOH to the reaction vessel. The reaction mixture was loaded on a plug of silica and eluted with EtOAc with 5% AcOH to produce the desired acid product after evaporation of chromotagraphy solvent in vacuo.

General Procedure (D) for the Synthesis of α-Deutero, α-Fluoro Carboxylic Acid:

To a flame dried 25 ml round bottom flask with magnetic stir bar was added triazolium salt XI (0.028 g, 0.066 mmol, 20 mol %). To the flask was added an α-fluoro aldehyde (0.060 g, 0.333 mmol, 1.0 eq) followed by toluene (0.02M with respect to aldehyde). The flask was purged with argon and brine (saturated solution of NaCl in D₂O) was added to the reaction vessel (of equal volume to 1.0 eq of 1M K₂CO₃ in D₂O). The reaction was stirred under an atmosphere of argon for 5 minutes followed by addition of 1.0 equiv of 1M K₂CO₃ in D₂O. The reaction was stirred until completion (8-10 hours) followed by addition of 1.0 equiv of AcOH to the reaction vessel. The reaction mixture was loaded on a plug of silica and eluted with EtOAc with 5% AcOH to produce the desired acid product after evaporation of chromotagraphy solvent in vacuo.

### Example 20

### Determination of Absolute Stereochemistry

The enantioenriched α-chloroester shown below can be accessed and its absolute stereochemistry determined by chemical correlation to an amino acid. This phenyl ester can be hydrolyzed to the α-chloro acid. HPLC elution indicates the same major enantiomer as that observed in the hydration reaction.

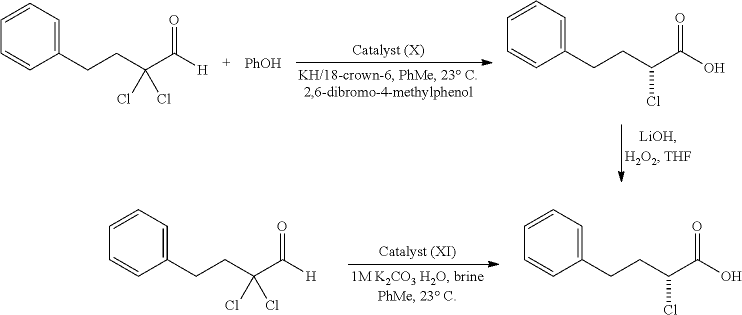

While not wishing to be held to theory, a model to account for absolute stereochemistry is shown below. Structure S-I is presumed to be the major olefin isomer generated as an intermediate. Chloride elimination affords S-II having the chloride substituent cis to the azolium. Protonation then occurs from the top face, controlled by the bulky indanyl group on the catalyst to afford S-IV.

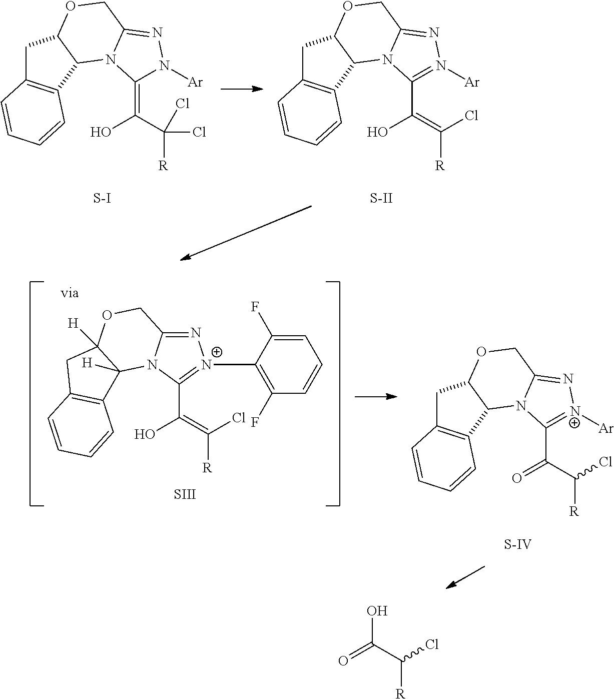

### Example 21

### Catalyst Preparation and Characterization

All catalysts were prepared according to literature procedure. During the synthesis of catalyst XIV it was noted that isolation and drying of the hydrazide was necessary to facilitate cyclization.

Triazolium Salt (XI): Rf=0.42 (EtOAc); [α]D 24=−179.8° (10 mg/ml, MeOH); mp=235° C.; ¹H NMR (400 MHz, acetone-d6) δ 11.10 (s, 1H), 7.90-7.87 (m, 1H), 7.65 (d, 1H, J=7.2 Hz), 7.50-7.32 (m, 5H), 6.30 (d, 1H, J=6.3 Hz), 5.37 (d, 1H, J=16 Hz), 5.24 (d, 1H, J=16.4 Hz), 5.20 (m, 1H); ¹³C NMR (100 MHz, acetone-d6) δ 158.1, 135.9 (t), 129.3 (d), 94.6, 77.6, 62.6, 60.2, 37.3; IR (KBr) 3133, 3115, 3072, 2955, 2916, 1627, 1605, 1588, 1540, 1484, 1210, 1067, 967 cm-1; HRMS (FAB+) calcd for C₁₈H₁₄F₂N₃O, 326.1105. Found 326.1102.

Triazolium Salt (XII): Rf=0.50 (EtOAc); [α]D 24=−165.0° (9 mg/ml, MeOH); mp=220° C.; ¹H NMR (400 MHz, acetone-d6) δ 11.37 (s, 1H), 8.73 (s, 2H), 8.42 (s, 1H), 7.70 (dd, 1H, J=7.6, 3.8 Hz), 7.45-7.40 (m, 2H), 7.32-7.28 (m, 1H), 6.22 (d, 1H, J=4.10 Hz), 5.83 (d, 1H, J=16.4 Hz), 5.22 (d, 1H, J=16.4 Hz), 5.14 (t, 1H, J=4.8 Hz), 3.54 (dd, 1H, J=17.2, 4.8 Hz), 3.26 (d, 1H, J=17.2 Hz); ¹³C NMR (100 MHz, acetone-d6) δ 151.1, 137.3 (q, J=34.6 Hz), 129.0 (br s), 123.0 (q, J=270 Hz), 121.6, 77.7, 62.5, 60.2, 37.3; IR (KBr) 3133, 3115, 3106, 2953, 2914, 1666, 1593, 1540, 1369, 1281, 1182, 1140, 899 cm-1; HRMS (FAB+) calcd for C₂₀H₁₃F₆N₃O, 426.1036. Found 426.1044.

Triazolium Salt (XIV): Rf=0.25 (EtOAc); [α]D 24=−63.3° (11 mg/ml, CH3CN); mp=260° C.; ¹H NMR (400 MHz, acetone-d6) δ 11.15 (s, 1H), 8.00 (s, 2H), 7.63-7.61 (m, 1H), 7.47-7.35 (m, 3H), 6.38 (d, 1H, J=4.10 Hz), 5.37 (d, 1H, J=16.4 Hz), 5.27 (d, 1H, J=16.4 Hz), 5.22 (t, 1H, J=4.8 Hz), 3.57 (dd, 1H, J=17.2, 4.8 Hz), 3.27 (d, 1H, J=17.2 Hz); ¹³C NMR (100 MHz, acetone-d6) δ 151.8, 127.0, 124, 77.6, 62.9, 60.2, 37.4; IR (KBr) 3124, 3089, 3068, 3050, 3007, 2911, 2855, 1588, 1566, 1536, 1466, 1414, 1149, 1054, 967 cm-1; HRMS (FAB+) calcd for C₁₈H₁₃Cl₃N₃O, 392.0019. Found 392.0124.

### Example 22

### Characterization of α,α-Dichloroaldehydes

General Comments: Retention factors (Rf) were unobtainable due to streaking of the compound on TLC plates. High-resolution mass spectra were also not obtainable due to decomposition of the aldehyde. The aldehydes are stable for several weeks stored in the neat form under Ar in a freezer.

### 2,2-dichloro-3-phenylpropanal (1a)

¹H NMR (400 MHz, CDC₃) δ 9.32 (s, 1H), 7.34 (s, 5H), 3.58 (s, 2H).

### 2,2-dichloro-3-(2-methoxyphenyl)propanal (1b)

¹H NMR (400 MHz, CDCl₃) δ 9.15 (s, 1H), 7.28-7.24 (m, 2H), 6.95-6.93 (m, 1H), 6.84-6.82 (m, 1H), 3.76 (s, 3H), 3.70 (s, 2H); ¹³C NMR (100 MHz, CDCl₃) δ 184.3, 133.0, 121.7, 89.0, 55.2, 43.2. IR (NaCl) 2941, 2840, 1750, 1491, 1524 cm-1.

### 2,2-dichloro-3-(4-tert butylaminophenyl)propanal (1c)

¹H NMR (400 MHz, CDCl₃) δ 9.28 (s, 1H), 7.32 (d, 2H, J=8.4 Hz), 7.25 (d, 1H, J=8.4 Hz), 6.48 (bs, 1H), 3.51 (s, 2H), 1.50 (s, 9H); NMR (100 MHz, CDCl₃) δ 185.6, 152.4, 87.7, 81.0, 45.8, 28.5; IR (NaCl) 3411, 3328, 2986, 2923, 1748, 1732, 1710, 1560, 1166 cm-1.

### 2,2-dichloro-3-cyclopentylpropanal (1d)

¹H NMR (400 MHz, CDCl₃) δ 9.18 (s, 1H), 2.32 (d, 2H, J=6.4 Hz), 2.11-2.0 (m, 1H), 1.87-1.80 (m, 2H), 1.60-1.43 (m, 4H), 1.16-1.01 (m, 2H); ¹³C NMR (100 MHz, CDCl₃) δ 185.4, 89.0, 46.5, 37.1, 33.9, 25.0; IR (NaCl) 2951, 2869, 1743, 1717 cm-1.

### 2,2-dichloro-3-cyclohexylpropanal (1e)

¹H NMR (400 MHz, CDCl₃) δ 9.21 (s, 1H), 2.24 (d, 2H, J=5.3 Hz), 1.81-1.61 (m, 6H), 1.32-1.00 (m, 5H).

### 3-benzyloxy-2,2-dichloropropanal (1f)

¹H NMR (400 MHz, CDCl₃) δ 9.28 (s, 1H), 7.35-7.24 (m, 5H), 4.65 (s, 2H), 4.02 (s, 2H); ¹³C NMR (100 MHz, CDCl₃) δ 185.0, 74.3; IR (NaCl) 3062, 3031, 2926, 2860, 1742, 1717, 1701, 1099 cm-1.

### 2,2-Dichloro-4-phenyl-butyraldehyde (1g)

¹H NMR (400 MHz) δ 9.31 (s, 1H), 7.28-7.40 (m, 5H), 3.00-3.07 (m, 2H), 2.63-2.67 (m, 2H).

### Methyl 6,6-dichloro-7-oxoheptanoate (1h)

¹H NMR (400 MHz, CDCl₃) δ 9.21 (s, 1H), 3.70 (s, 3H), 2.25-2.40 (m, 4H), 1.65-1.71 (m, 4H). ¹³C NMR (100 MHz, CDCl₃) δ 184.8, 94.2, 88.4, 51.8, 40.3, 33.8, 24.2; IR (NaCl) 2955, 2869, 1438 cm-1.

### Example 23

### Characterization of α-Chloro,α-Proteo and α-Chloro,α-Deutero Carboxylic Acids

(R)-2-chloro-3-phenylpropionic acid (2a; X=H): Title compound was prepared according to general procedure A. Rf=0.32 (8:2 Hexanes:EtOAc w/3% AcOH); [α]D 24=−6.8° (10 mg/ml, MeOH); HPLC—analysis Chiracel OJH column 95:5 hexanes:isopropanol 1 ml/min for 30 min. Major: 13.69 min, Minor: 19.21 min; ¹H NMR (400 MHz, CDCl₃) δ 10.80 (bs, 1H), 7.17-7.29 (m, 5H), 4.50 (t, 1H, J=10 Hz), 3.40 (dd, 1H, J=8.8, 19.2 Hz), 3.20 (dd, 1H, J=10.4, 18.8 Hz).

(R)-2-deutero, chloro-3-phenylpropanoic acid (2a; X=D): Title compound was prepared according to general procedure B. Rf=0.32 (8:2 Hexanes:EtOAc w/3% AcOH); [α]D 24=−6.7° (13 mg/1 ml, MeOH); HPLC—analysis Chiracel OJ-H column 95:5 hexanes:isopropanol 1 ml/min for 30 min. Major: 13.94 min, Minor: 19.50 min; ¹H NMR (400 MHz, CDCl₃) δ 9.10 (s, 1H), 7.16-7.28 (m, 5H), 3.30 (bd, 1H, J=14.4 Hz), 3.10 (bd, 1H, J=14.4 Hz); ¹³C NMR (100 MHz, CDCl₃) δ 174.0, 129.6, 58.9 (t), 41.0; IR (NaCl) 3133, 3115, 3072, 2955, 2916, 1627, 1605, 1588, 1540, 1484, 1210, 1067, 967 cm-1; HRMS (FAB+) calcd for C₉H₈ClO₂, 184.0281. Found 184.0288.

(R)-2-chloro-3-(methoxyphenyl)propanoic acid (2b; X=H): Rf=0.22 (7:3 Hexanes:EtOAc); [α]D 24=−31.6° (10 mg/ml, MeOH); HPLC—analysis Chiracel OD-H column 99:1 hexanes:isopropanol 1 ml/min for 30 min. Major: 14.20 min, Minor: 10.52 min; ¹H NMR (400 MHz, CDCl₃) δ 10.04 (bs, 1H), 7.28-7.15 (m, 2H), 6.91-6.84 (m, 2H), 4.65 (t, 1H, J=7.4 Hz), 3.81 (s, 3H), 3.39 (dd, 1H, J=13.6, 7.2 Hz), 3.16 (dd, 1H, J=14, 8.0 Hz); ¹³C NMR (100 MHz, acetone-d6) δ 175.0, 129.5, 55.6, 55.4, 36.7, 30.0; IR (NaCl) 3133, 3115, 3072, 2955, 2916, 1627, 1605, 1588, 1540, 1484, 1210, 1067, 967 cm-1; HRMS (FAB+) calcd for C₁₀H₁₀ClO₃, 213.0324. Found 213.0325.

(R)-2-deutero, chloro-3-(methoxyphenyl)propanoic acid (2b; X=D): Rf=0.22 (7:3 Hexanes:EtOAc); [α]D 24=−35.0° (10 mg/ml, MeOH); HPLC—analysis Chiracel OD-H column 99:1 hexanes:isopropanol 1 ml/min for 30 min. Major: 16.33 min, Minor: 11.87 min; ¹H NMR (400 MHz, CDCl₃) δ 9.67 (bs, 1H), 7.27-7.15 (m, 2H), 6.90-6.83 (m, 2H), 3.81 (s, 3H), 3.40 (bd, 1H, J=13.6 Hz), 3.18 (bd, 1H, J=13.6 Hz); ¹³C NMR (100 MHz, CDCl₃) δ 175.6, 157.5, 55.6, 55.4, 36.7, 30.0; IR (NaCl) 3133, 3115, 3072, 2955, 2916, 1627, 1605, 1588, 1540, 1484, 1210, 1067, 967 cm-1; HRMS (FAB+) calcd for C₁₀H₉ClO₃, 214.0387. Found 214.0389.

(R)-2-chloro-3-(4-tert-butoxycarbonylaminophenyl)propanoic acid (2c; X=H): Title compound was prepared according to general procedure A.Rf=0.42 (1:1 Hexanes:EtOAc); [α]D 24=−74.8° (10 mg/ml, MeOH); HPLC—analysis Chiracel OD-H column 95:5 hexanes:isopropanol 1 ml/min for 50 min. Major: 43.56 min, Minor: 37.86 min; ¹H NMR (400 MHz, CDCl₃) δ 8.58 (bs, 1H), 7.23 (d, 2H, J=8.4 Hz), 7.12 (d, 2H, J=8.4 Hz), 6.68 (bs, 1H), 4.42 (t, 1H, J=7.2 Hz), 3.29 (dd, 1H, J=14.0, 7.2 Hz), 3.13 (dd, 1H, J=14.0, 7.2 Hz), 1.50 (s, 9H); ¹³C NMR (100 MHz, CDCl₃) δ 173.1, 57.5, 40.5, 28.5; IR (NaCl) 3331, 2980, 2931, 2627, 2360, 1715, 1614, 1596, 1524, 1414, 1393, 1369, 1159 cm-1; HRMS (FAB+) calcd for C₁₄H₁₇ClNO₄, 298.0852. Found 298.0855.

(R)-2-deutero, chloro-3-(4-tert-butoxycarbonylaminophenyl)propanoic acid (2c; X=D): Title compound was prepared according to general procedure B. Rf=0.42 (1:1 Hexanes:EtOAc); [α]D 24=−78.9° (10 mg/ml, MeOH); HPLC—analysis Chiracel OD-H column 95:5 hexanes:isopropanol 1 ml/min for 50 min. Major: 43.0 min, Minor: 37.60 min; ¹H NMR (400 MHz, CDCl₃) δ 7.23 (d, 2H, J=8.0 Hz), 7.12 (d, 2H, J=8.0 Hz), 3.29 (bd, 1H, J=14.0 Hz), 3.12 (bd, 1H, J=14.0 Hz), 1.50 (s, 9H); ¹³C NMR (100 MHz, CDCl₃) δ 173.7, 130.0, 58.6 (t), 40.4, 28.5; IR (NaCl) 3331, 2980, 2931, 2627, 2360, 1715, 1614, 1596, 1524, 1414, 1393, 1369, 1159 cm-1; HRMS (FAB+) calcd for C₁₄H₁₆DClNO₄, 299.0914. Found 299.092.

(R)-2-chloro-3-cyclopentylpropanoic acid (2d; X=H): Title compound was prepared according to general procedure A. Rf=0.22 (1:1 Hexanes:EtOAc); [α]D 24=−95.0° (10 mg/ml, MeOH); GC—analysis Chiral BDM-1 column, 140° C., 1 ml/min for 45 min. Major: 7.58 min, Minor: 5.77 min; ¹H NMR (400 MHz, CDCl₃) δ 9.77 (bs, 1H), 4.29 (t, 1H, J=7.6 Hz), 1.95-2.03 (m, 3H), 1.80-1.84 (m, 2H), 1.52-1.63 (m, 4H), 1.06-1.16 (m, 2H); ¹³C NMR (100 MHz, CDCl₃) δ 176.0, 56.8, 41.1, 37.1, 32.6, 32.1, 25.3, 25.1; IR (NaCl) 2951, 2869, 1721, 1431, 1289, 1205 cm-1; HRMS (FAB+) calcd for C₈H₁₂ClO₂, 175.0531. Found 175.0532.

(R)-2-deutero, chloro-3-cyclopentylpropanoic acid (2d; X=D): Title compound was prepared according to general procedure B. Rf=0.24 (1:1 Hexanes:EtOAc); [α]D 24=−98.5° (10 mg/ml, MeOH); GC—analysis Chiral BDM-1 column, 140° C., 1 ml/min for 46 min. Major: 7.58 min, Minor: 5.70 min; ¹H NMR (400 MHz, CDCl₃) δ 10.24 (bs, 1H), 1.95-2.02 (m, 3H), 1.78-1.87 (m, 2H), 1.53-1.64 (m, 4H), 1.12-1.19 (m, 2H); ¹³C NMR (100 MHz, CDCl₃) δ 176.2, 56.4 (t), 41.0, 37.1, 32.6, 32.1, 31.3, 25.2, 25.0; IR (NaCl) 3109, 2952, 2869, 2670, 2554, 1720, 1451, 1414, 1284, 1205 cm-1; HRMS (FAB+) calcd for C₈H₁₁DClO₂, 176.0594. Found 176.0594.

(R)-2-chloro-3-cyclohexylpropanoic acid (2e; X=H): Title compound was prepared according to general procedure A. Rf=0.20 (1:1 Hexanes:EtOAc); [α]D 24=−105.1° (10 mg/ml, MeOH); GC—analysis Chiral BDM-1 column, 100° C., 1 ml/min for 120 min. Major: 85.92 min, Minor: 88.61 min; ¹H NMR (400 MHz, CDCl₃) δ 9.67 (bs, 1H), 4.36 (dd, 1H, J=6.0, 8.8 Hz), 1.80-1.92 (m, 2H), 1.63-1.74 (m, 5H), 1.46-1.59 (m, 1H), 1.11-1.30 (m, 3H), 0.82-1.2 (m, 2H); ¹³C NMR (100 MHz, CDCl₃) δ 176.0, 55.2, 42.2, 34.5, 33.4, 32.1, 26.5, 26.2, 26.1; IR (NaCl) 3109, 2925, 2853, 2672, 1723, 1449, 1429, 1291, 1211 cm-1; HRMS (FAB+) calcd for C₉H₁₄ClO₂, 189.0688. Found 189.0688.

(R)-2-chloro-3-cyclohexylpropanoic acid (2e; X=D): Title compound was prepared according to general procedure B. Rf=0.2 (1:1 Hexanes:EtOAc); [α]D 24=−108.2° (10 mg/ml, MeOH); GC—analysis Chiral BDM-1 column, 100° C., 1 ml/min for 120 min. Major: 85.92 min, Minor: 88.61 min; ¹H NMR (400 MHz, CDCl₃) δ 10.21 (bs, 1H), 1.74-1.90 (m, 2H), 1.64-1.74 (m, 5H), 1.47-1.56 (m, 1H), 1.11-1.30 (m, 3H), 0.82-1.2 (m, 2H); ¹³C NMR (100 MHz, CDCl₃) δ 176.2, 54.6 (t), 42.1, 34.5, 33.4, 32.1, 26.5, 26.2, 26.1; IR (NaCl) 3098, 3036, 2925, 2853, 2669, 2537, 2342, 2360, 1721, 1449, 1415, 1292, 1282, 1213 cm-1; HRMS (FAB+) calcd for C₉H₁₃DClO₂, 190.0751. Found 190.0752.

(R)-2-chloro-3-cyclohexylpropanoic acid (2f; X=H): Title compound was prepared according to general procedure A. Rf=0.35 (7:3 Hexanes:EtOAc); [α]D 24=−27.3° (10 mg/ml, MeOH); HPLC—analysis Chiracel OD-H column 99:1 hexanes:isopropanol 1 ml/min for 10 min. Major: 8.28 min, Minor: 8.74 min 1 ml/min for 10 min; ¹H NMR (400 MHz, CDCl₃) δ 9.10 (bs, 1H), 7.30-7.33 (m, 5H), 4.60 (s, 2H), 4.30 (dd, 1H, J=5.6, 10.4 Hz), 3.89 (dd, 1H, J=8.4, 10.4 Hz), 3.83 (dd, 1H, J=6.0, 10.4 Hz); ¹³C NMR (100 MHz, CDCl₃) δ 137.0, 73.9, 71.0; IR (NaCl) 3439, 3165, 3032, 2926, 2871 2644, 2582, 1733, 1453, 1107 cm-1; HRMS (FAB+) calcd for C₁₀H₁₀ ClO₃, 213.0324. Found 213.0321.

(R)-2-deutero, chloro-3-cyclohexylpropanoic acid (2f; X=D): Title compound was prepared according to general procedure B. Rf=0.32 (1:1 Hexanes:EtOAc); [α]D 24=−30.1° (10 mg/ml, MeOH); HPLC—analysis Chiracel OD-H column 99:1 hexanes:isopropanol 1 ml/min for 10 min. Major: 8.32 min, Minor: 8.84 min 1 ml/min for 10 min; ¹H NMR (400 MHz, CDCl₃) δ 9.10 (bs, 1H), 7.24-7.35 (m, 5H), 4.60 (s, 2H), 3.89 (bd, 1H, J=10.0 Hz), 3.83 (bd, 1H, J=10.0 Hz); ¹³C NMR (100 MHz, CDCl₃) δ 172.0, 127.6, 127.9, 73.9, 70.9, 53.4 (t), 27.7; IR (NaCl) 3176, 3086, 3064, 2929, 2868, 2801, 1726, 1496, 1453, 1237, 1213 cm-1; HRMS (FAB+) calcd for C₁₀H₉ClO₃, 214.0387. Found 214.0392.

(R)-2-deutero, chloro-3-cyclohexylpropanoic acid (2g; X=H): Title compound was prepared according to general procedure A. Rf=0.45 (7:3 Hexanes:EtOAc); [α]D 24=−25.8° (10 mg/ml, MeOH); HPLC—analysis Chiracel OJ-H column 90:10 hexanes:isopropanol 1 ml/min for 30 min. Major: 8.32 min, Minor: 8.84 min 1 ml/min for 10 min; ¹H NMR (400 MHz, CDCl₃) δ 7.26 (bs, 1H), 7.13-7.24 (m, 5H), 4.20 (dd, 1H, J=4.8, 8.4 Hz), 2.70-2.80 (m, 2H), 2.16-2.30 (m, 2H); ¹³C NMR (100 MHz, CDCl₃) δ 175.7, 56.4, 36.4, 32.1; IR (NaCl) 3031, 3086, 3064, 2929, 2868, 2801, 1721, 1496, 1453, 1237, 1213 cm-1; HRMS (FAB+) calcd for C₁₀H₁₀ClO₂, 197.0375. Found 197.0376.

(R)-2-deutero, chloro-3-cyclohexylpropanoic acid (2g; X=D): Title compound was prepared according to general procedure B. Rf=0.45 (7:3 Hexanes:EtOAc); [α]D 24=−27.2° (10 mg/ml, MeOH); HPLC—analysis Chiracel OD-H column 99:1 hexanes:isopropanol 1 ml/min for 10 min. Major: 8.32 min, Minor: 8.84 min 1 ml/min for 10 min; ¹H NMR (400 MHz, CDCl₃) δ 9.15 (bs, 1H), 7.13-7.26 (m, 5H), 2.68-2.81 (m, 2H), 2.16-2.33 (m, 2H); ¹³C NMR (100 MHz, CDCl₃) δ 175.7, 56.1 (t), 36.3, 32.1; IR (NaCl) 3176, 3086, 3064, 2929, 2868, 2801, 1726, 1496, 1453, 1237, 1213 cm-1; HRMS (FAB+) calcd for C₁₀H₉ClO₂, 198.0438. Found 198.0438.

(R)-2-chloro-7-methoxy-7-oxoheptanoic acid (2h; X=H): Title compound was prepared according to general procedure A. Rf=0.18 (1:1 Hexanes:EtOAc); [α]D 24=−89.1° (10 mg/ml, MeOH); HPLC—analysis Chiracel AD-H column 95:5 hexanes:isopropanol 1 ml/min for 50 min. Major: 46.4 min, Minor: 48.3 min; ¹H NMR (400 MHz, CDCl₃) δ 9.05 (bs, 1H), 4.30 (bs, 1H), 3.65 (s, 3H), 3.32 (t, 2H, J=7.2 Hz), 1.95-2.04 (m, 2H), 1.50-1.66 (m, 4H); ¹³C NMR (100 MHz, acetone-d6) δ 175.1, 59.8, 51.9, 34.5, 33.9, 25.6, 24.7, 24.3; IR (NaCl) 3467, 2954, 2869, 2360, 1732, 1636, 1457, 1439, 1368, 1177 cm-1; HRMS (FAB+) calcd for C₈H₁₂ClO₄, 207.043. Found 207.0431.

(R)-2-deutero, chloro-7-methoxy-7-oxoheptanoic acid (2h; X=D): Title compound was prepared according to general procedure B. Rf=0.17 (1:1 Hexanes:EtOAc); [α]D 24=−92.5° (10 mg/ml, MeOH); HPLC—analysis Chiracel AD-H column 95:5 hexanes:isopropanol 1 ml/min for 60 min. Major: 47.3 min, Minor: 49.6 min; ¹H NMR (400 MHz, CDCl₃) δ 10.17 (bs, 1H), 4.30 (bs, 1H), 3.65 (s, 3H), 2.28-2.36 (m, 2H), 2.01-2.07 (m, 1H), 1.89-2.00 (m, 1H), 1.58-1.70 (m, 3H), 1.43-1.57 (m, 2H), 1.47-1.56 (m, 2H); ¹³C NMR (100 MHz, CDCl₃) δ 174.5, 56.9 (t), 51.9, 34.4, 33.9, 25.5, 24.6; IR (NaCl) 3467, 3182, 2953, 2868, 2633, 2537, 2360, 1737, 1720, 1457, 1439, 1367, 1223, 1169 cm-1; HRMS (FAB+) calcd for C₈H₁₁DClO₄, 208.0492. Found 208.0496.

### Example 24

### Synthesis of α-Fluoro Enals

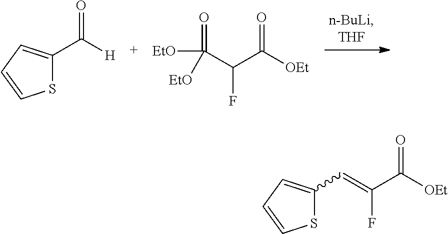

A flame dried 25 ml round bottom flask with stir bar was charged with 2-Fluoro-2-phosphonoacetic acid triethyl ester (1.50 g, 6.19 mmol, 1.0 eq.), and placed under an argon atmosphere. Anhydrous THF (20 ml) was then added to the reaction vessel and the clear solution was agitated and cooled to 0° C. 4 ml of n-BuLi (1.6 M in hexanes) was slowly added to the reaction vessel. Upon complete addition the vessel was warmed to 25° C. and stirred for 30 min. 2-Thiophene carboxaldehyde was the added to the reaction vessel and stirred for 12 hrs. The reaction was quenched at 0° C. via the addition of 15 ml of 10% HCl. The mixture was extracted with EtOAc and washed with water and brine. The organic solution was dried with magnesium sulfate and filtered to yield a crude yellow solution, which was reduced in vacuo to yield a yellow oil. The oil is purified via column chromatography in 97:3 hexanes:ethyl acetate to 95:5 hexanes:ethyl acetate to yield the desired product as a mixture of olefin isomers in an 80% yield.

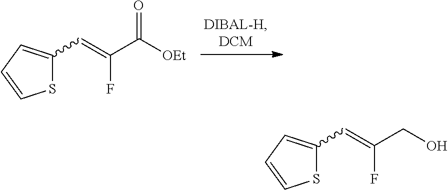 [SMILES: CCOC(=O)C(F)=Cc1cccs1 ; OCC(F)=Cc1cccs1]

A flame dried 25 ml round bottom flask with stir bar was charged with ester (0.934 g, 4.66 mmol, 1.0 eq). To the flask was added 15 ml of anhydrous DCM and the solution stirred under an argon atmosphere. To the reaction vessel was added DIBAl—H (1M in PhMe, 18.64 ml, 3.0 eq) at 25° C. The reaction was stirred until consumption of starting material was observed by TLC; the reaction vessel was then cooled to 0° C. and 15 ml of a saturated solution of Rochelle's salt was added and the vessel was allowed to warm to 25° C. The solution was stirred until a phase separation was observed. The solution was extracted with DCM and washed with water and brine. The organic solution was dried with magnesium sulfate, filtered and reduced in vacuo to yield an oil. The oil was purified by column chromatography in 8:2 hexanes:ethyl acetate to yield the alcohol in 75% yield.

¹H NMR (400 MHz, CDCl₃) δ 7.25 (dd, 1H, J=1.2, 5.2 Hz), 6.92-6.99 (m, 2H), 6.42 (d, 1H, J=18.8 Hz), 4.49 (d, 2H, J=21.6 Hz); ¹³C NMR (100 MHz, CDCl₃) δ 174.9, 174.5, 56.9 (t), 51.9, 34.4, 33.9, 25.5, 24.6;

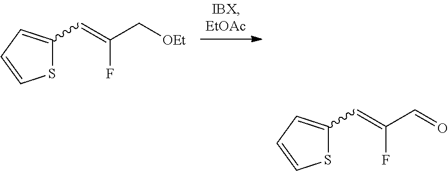 [SMILES: CCOCC(F)=Cc1cccs1 ; O=CC(F)=Cc1cccs1]

A 100 ml round bottom was charged with alcohol (0.50 g, 1.0 eq) and dissolved in 40 ml of EtOAc. IBX (2.21 g, 2.5 eq) was added to the flask. A reflux condenser was fitted to the flask and the heterogeneous mixture was stirred and heated at 70° C. until consumption of alcohol was observed by TLC. The flask was cooled to 25° C. and then cooled to 0° C. for 2 hours. The heterogeneous mixture was filtered over a pad of celite. The pad was further washed with cold EtOAc and the solution was reduced in vacuo to yield the desired aldehyde (80% yield).

### Example 25

### Characterization of α-Fluoro Enals

General Comments: High-resolution mass spectra were not attainable due to decomposition of the aldehyde. The aldehydes were stored under Ar in the freezer for several days. Decomposition is observed if the aldehyde is stored at ambient temperature or upon prolonged exposure to light.

(E/Z)-2-fluoro-3-(4-methoxyphenyl)acrylaldehyde (3a): Rf=0.24 (9:1 Hexanes:EtOAc); ¹H NMR (400 MHz, CDCl₃) δ 9.64 (d, 1H, J=20.4 Hz), 4.30 (bs, 1H), 7.31 (d, 2H, J=7.6 Hz), 7.23 (d, 1H, J=1.2 Hz), 6.92 (d, 2H, J=8.0 Hz), 3.81 (s, 3H; ¹³C NMR (100 MHz, CDCl₃) δ 183.0 (d), 131.7, 55.6; IR (NaCl) 3008, 2958, 2937, 2841, 2562, 2361, 2052, 1683, 1629, 1604, 1570, 1511, 1464, 1443, 1331, 1302, 1239, 1171, 1029 cm-1;

(E/Z)-2-fluoro-3-(2-methoxyphenyl)acrylaldehyde (3b): Rf=0.21 (9:1 Hexanes:EtOAc); ¹H NMR (400 MHz, CDCl₃) δ 9.32 (d, 1H, J=18 Hz), 7.37-7.42 (m, 2H), 6.90-7.02 (m, 3H), 3.89 (s, 3H); ¹³C NMR (100 MHz, CDCl₃) δ 184.3 (d), 132.7 (d), 121.3, 111.1, 55.8; IR (NaCl) 3011, 2946, 2841, 1683, 1627, 1598, 1540, 1488, 1465, 1437, 1374, 1328, 1291, 1254, 1227 cm-1;

(E/Z)-2-fluoro-3-(naphthalene-1-yl)acrylaldehyde (3c): Rf=0.31 (9:1 Hexanes:EtOAc); ¹H NMR (400 MHz, CDCl₃) δ 9.52 (d, 1H, J=19.6 Hz), 7.86-7.94 (m, 4H), 7.44-7.59 (m, 4H); ¹³C NMR (100 MHz, CDCl₃) δ 183.1 (d), 130.0, 129.0, 127.0, 126.1, 125.8; IR (NaCl) 3059, 2956, 2868, 1949, 1777, 1692, 1664, 1642, 1509, 1462, 1324, 1271, 1242, 1215, 1195 cm-1;

(E/Z)-2-fluoro-3-(thiophen-2-yl)acrylaldehyde (3d): Rf=0.42 (9:1 Hexanes:EtOAc); ¹H NMR (400 MHz, CDCl₃) δ 9.28 (d, 1H, J=19.2 Hz), 7.46-7.48 (m, 1H), 7.26-2.27 (m, 1H), 7.06-7.10 (m, 1H), 6.92 (d, 1H, J=33.2 Hz); ¹³C NMR (100 MHz, CDCl₃) δ 182.2 (d), 152.0 (d), 133.3 (d); IR (NaCl) 3108, 2861, 1674, 1653, 1646, 1558, 1540, 1320, 1245, 1224, 885 cm-1;

(E/Z)-2-fluoro-3-cyclohexyl acrylaldehyde (3e): Rf=0.31 (8:2 Hexanes:EtOAc); ¹H NMR (400 MHz, CDCl₃) δ 9.13 (d, 1H, J=18 Hz), 5.76 (dd, 1H, J=9.6, 33.6 Hz), 2.66 (d, 1H, J=10.4 Hz), 1.65-1.75 (m, 5H), 1.10-1.37 (m, 5H); ¹³C NMR (100 MHz, CDCl₃) δ 184.2 (d), 155.0 (d), 136.4 (d), 34.6, 32.0, 25.8, 25.5; IR (NaCl) 3467, 3182, 2953, 2868, 2633, 2537, 2360, 1737, 1720, 1457, 1439, 1367, 1223, 1169 cm-1.

### Example 26

### Characterization of α-Fluoro Carboxylic Acids

(R)-2-fluoro-3-(4-methoxyphenyl)propanoic acid (4a): Title compound was prepared according to general procedure C. Rf=0.23 (8:2 Hexanes:EtOAc); [α]D 24=−32.4° (10 mg/ml, MeOH); HPLC—analysis Chiracel OD-H column 99:1 hexanes:isopropanol 1 ml/min for 60 min. Major: 54.3 min, Minor: 57.6 min; ¹H NMR (400 MHz, CDCl₃) δ 7.16 (d, 2H, J=8.8 Hz), 6.83 (d, 2H, J=8.4 Hz), 5.10 (ddd, 1H, J=4.0, 7.6, 48.8 Hz), 3.22 (ddd, 1H, J=4.0, 14.8, 28.0 Hz), 3.11 (ddd, 1H, J=7.6, 14.8, 25.6 Hz); ¹³C NMR (100 MHz, CDCl₃) δ 174.2 (d), 130.3, 55.5 (d), 37.7 (d); IR (NaCl) 2935, 2837, 1777, 1733, 1700, 1684, 1675, 1652, 1646, 1635, 1575, 1514, 1248 cm-1; HRMS (FAB+) calcd for C₁₀H₁₀FO₃, 197.0619. Found 197.0619.

(R)-2-fluoro-3-(2-methoxyphenyl)propanoic acid (4b): Title compound was prepared according to general procedure C. Rf=0.20 (8:2 Hexanes:EtOAc); [α]D 24=−40.3° (10 mg/ml, MeOH); HPLC—analysis Chiracel OD-H column 95:5 hexanes:isopropanol 1 ml/min for 60 min. Major: 41.3 min, Minor: 45.6 min; ¹H NMR (400 MHz, CDCl₃) δ 7.23-7.27 (m, 1H), 7.16-7.18 (m, 1H), 6.85-6.91 (m, 2H), 5.22 (ddd, 1H, J=4.4, 8.4, 48.8 Hz), 3.38 (ddd, 1H, J=4.4, 14.4, 30.0 Hz), 3.10 (ddd, 1H, J=8.4, 14.4, 18.4 Hz); ¹³C NMR (100 MHz, CDCl₃) δ 174.8 (d), 157.5, 55.4, 33.8 (d); IR (NaCl) 2940, 2839, 1733, 1602, 1495, 1465, 1439, 1247 cm-1; HRMS (FAB+) calcd for C₁₀H₁₀FO₃, 197.0619. Found 197.0623.

(R)-2-fluoro-3-(naphthalene-1-yl)propanoic aicd (4c): Title compound was prepared according to general procedure C. Rf=0.3 (7:3 Hexanes:EtOAc); [α]D 24=−28.3° (10 mg/ml, MeOH); HPLC—analysis Chiracel OJ-H column 95:5 hexanes:isopropanol 1 ml/min for 60 min. Major: 42.0 min, Minor: 43.1 min; ¹H NMR (400 MHz, CDCl₃) δ 8.02 (d, 1H, J=8.8 Hz), 7.87 (d, 1H, J=7.6 Hz), 7.79-7.81 (m, 1H), 7.48-7.58 (m, 2H), 7.42-7.43 (m, 2H), 5.28 (ddd, 1H, J=3.2, 8.8, 48.8 Hz), 3.82 (ddd, 1H, J=3.6, 15.2, 31.2 Hz), 3.55 (ddd, 1H, J=8.8, 15.2, 20.4 Hz); ¹³C NMR (100 MHz, CDCl₃) δ 175.1 (d), 134.5, 128.0, 125.3, 89.6, 87.7, 35.8 (d); IR (NaCl) 3057, 2931, 1718, 1700, 1684, 1675, 790, 775 cm-1; HRMS (FAB+) calcd for C₁₃H₁₀FO₂, 217.067. Found 217.0675.

(R)-2-fluoro-2-thiophen-2-yl)propanoic acid (4d): Title compound was prepared according to general procedure C. Rf=0.44 (6:4 Hexanes:EtOAc); [α]D 24=−93.4° (10 mg/ml, MeOH); HPLC—analysis Chiracel AD-H column 95:5 hexanes:isopropanol 1 ml/min for 60 min. Major: 35.6 min, Minor: 41.1 min; ¹H NMR (400 MHz, CDCl₃) δ 7.19-7.23 (m, 1H), 6.94-6.96 (m, 2H), 5.18 (ddd, 1H, J=4.0, 6.8, 48.4 Hz), 3.35-3.55 (m, 2H); ¹³C NMR (100 MHz, CDCl₃) δ 174.5 (d), 127.4, 89.3, 87.3, 32.8 (d); IR (NaCl) 3108, 2974, 1700, 1684, 1456, 1436, 1418, 701 cm-1; HRMS (FAB+) calcd for C₇H₆FO₂S, 173.0078. Found 173.0081.

(R)-2-fluoro-3-cyclohexyl propanoic acid (4e): Title compound was prepared according to general procedure C. Rf=0.2 (1:1 Hexanes:EtOAc); [α]D 24=−115.6° (10 mg/ml, MeOH); GC—analysis Chiral BDM-1 column, 100° C., 1 ml/min for 120 min. Major: 80.62 min, Minor: 83.61 min; ¹H NMR (400 MHz, CDCl₃) δ 5.02 (ddd, 1H, J=3.6, 9.2, 49.6 Hz), 1.55-1.86 (m, 8H), 0.91-1.27 (m, 5H); ¹³C NMR (100 MHz, CDCl₃) δ 176.2 (d), 87.0 (d), 39.7 (d), 33.8, 32.4, 32.0, 26.5, 26.2, 26.1, 25.9, 25.5; IR (NaCl) 3010, 2925, 2852, 1733, 1684, 1652, 1448, 1276, 1232, 1097, 1074 cm-1; HRMS (FAB+) calcd for C₉H₁₄FO₂, 173.0983. Found 173.0988.

### Example 27

### Characterization of α-Fluoro,α-Deutero Carboxylic Acid

(R)-2-deutero, fluoro-3-(4-methoxyphenyl)propanoic aicd (6a): Title compound was prepared according to general procedure D. Rf=0.18 (8:2 Hexanes:EtOAc); [α]D 24=−36.0° (10 mg/ml, MeOH); HPLC—analysis Chiracel OD-H column 99:1 hexanes:isopropanol 1 ml/min for 60 min. Major: 43.6 min, Minor: 48.4 min; ¹H NMR (400 MHz, CDCl₃) δ 7.15 (d, 2H, J=8.4 Hz), 6.84 (d, 2H, J=11.2 Hz), 3.77 (s, 3H), 3.08-3.25 (m, 2H); ¹³C NMR (100 MHz, CDCl₃) δ 175.3 (d), 159.0, 131.3, 80.1 (dt), 55.4, 37.6 (d); IR (NaCl) 3041, 2933, 2838, 1734, 1653, 1558, 1539, 1513, 1250, 1180, 800, 778 cm-1; HRMS (FAB+) calcd for C₁₀H₉DFO₃, 198.0682. Found 198.068.

It is understood for purposes of this disclosure, that various changes and modifications may be made to the invention that are well within the scope of the invention. Numerous other changes may be made which will readily suggest themselves to those skilled in the art which are encompassed in the spirit of the invention disclosed herein and as defined in the appended claims.

As used in the specification and the appended claims, the singular forms “a”, “an”, and “the” include plural referents unless the context clearly dictates otherwise. Thus, for example, reference to “a compound” encompasses a combination or mixture of different compounds as well as a single compound, reference to “a solvent” includes a single solvent as well as solvent mixture, and the like.

This specification contains numerous citations to references such as patents, patent applications, and publications. Each is hereby incorporated by reference for all purposes.

The following publications are individually incorporated by reference herein:
Ueda, M.; Kano, T.; Maruoka, K. Org. Biomol. Chem. 2009, 7, 2005-2012.
Marigo, M.; Jørgensen, K. A. Chem. Commun. 2006, 2001-2011.
Guillena, G.; Ramon, D. J. Tetrahedron Asym. 2006, 17, 1465-1492.
France, S.; Weatherwax, A.; Lectka, T. Eur. J. Org. Chem. 2005, 475-479.
Ostereich, M. Angew. Chem. Int. Ed. 2005, 44, 2324-2327.
Ibrahim, H.; Togni, A. Chem. Commun. 2004, 1147-1155.
Muñiz, K. Angew. Chem. Int. Ed. 2001, 40, 1653-1655.
Amatore, M.; Beeson, T. D.; Brown, S. P.; MacMillan, D. W. C. Angew. Chem. Int. Ed. 2009, 48, 5121-5124.
Brochu, M. P.; Brown, S. P.; MacMillan, D. W. C. J. Am. Chem. Soc. 2004, 126, 4108-4109.
Halland, N.; Braunton, A.; Bachmann, S.; Marigo, M.; JØrgensen, K. A. J. Am. Chem. Soc. 2004, 126, 4790-4791.
Halland, N.; Lie, M. A.; Kjaersgaard, A; Marigo, M.; Schiøtt, B.; Jørgensen, K. A. Chem. Eur. J. 2005, 11, 7083-7090.
France, S.; Wack, H.; Tagi, A. E.; Hafez, A. M.; Wagerle, T. R.; Shah, M. H.; Dusich, C. L.; Lectka, T. J. Am. Chem. Soc. 2004, 126, 4245-4255.
Lee, E. C.; McCauley, K. M.; Fu, G. C. Angew. Chem. Int. Ed. 2007, 46, 977-979.
Moore, J. L.; Rovis, T. Top. Curr. Chem. 2009, 291, DOI: 10.1007/128_{—}2008_{—}18.
Enders, D.; Niemeier, 0.; Hensler, A. Chem. Rev. 2007, 107, 5606-5655.
Reynolds, N. T.; Rovis, T. J. Am. Chem. Soc. 2005, 127, 16406-16407.
Reynolds, N. T.; Read de Alaniz, J.; Rovis, T. J. Am. Chem. Soc. 2004, 126, 9518-9519.
Chow, K. Y.-K.; Bode, J. W. J. Am. Chem. Soc. 2004, 126, 8126-8127.
Mohr, J. T.; Hong, A. Y.; Stoltz, B. M. Nature Chem. 2009, 1, 359-369.
Burns, N. Z.; Baran, P. S.; Hoffmann, R. W. Angew. Chem. Int. Ed. 2009, 48, 2854-2867.
Sohn, S. S.; Bode, J. W. Angew. Chem. Int. Ed. 2006, 45, 6021-6024.
He, M.; Beahm, B. J.; Bode, J. W. Org. Lett. 2008, 10, 3817-3820.
Amyes, T. L.; Diver, S. L.; Richard, J. P.; Rivas, F. M.; Toth, K. J. Am. Chem. Soc. 2004, 126, 4366-4374.
Takikawa, H.; Suzuki, K. Org. Lett. 2007, 9, 2713-2716.
Becker, H. G. 0.; Gwam, K. M. J. Prakt. Chem. 1992, 334, 231-236.
de Alaniz, J.; Ken, M. S.; Moore, J. L.; Rovis, T. J. Org. Chem. 2008, 73, 2033-2040.
Yarnell, A. T. Chem. Eng. News 2009, 87, 36-39.
Böhm, H.; Banner, D.; Bendels, S.; Kansy, M.; Kuhn, B.; Obst-Sander, U.; Stahl, M. ChemBioChem 2004, 5, 637.
Maienfisch, P.; Hall, R. G.; Chimia 2004, 58, 93.
Hiyama, T.; Shimizu, M. Angew. Chem. Int. Ed. 2005, 44, 214.
Purser, S.; Moore, P. R.; Swallow, S.; Gouverneur, V. Chem. Soc. Rev. 2008, 37, 320-330.
Hagmann, W. K. J. Med. Chem. 2008, 51, 4359-4369.
Barber, J.; Keck, R.; Rétey, J. Tetrahedron Lett. 1982, 23, 1549-1552.
Takeuchi, Y.; Nagata, K.; Koizumi, T. J. Org. Chem. 1989, 54, 5453-5459.
Gritz-Langhals, E.; Schutz, G. Tetrahedron Lett. 1993, 34, 293-296.

What is claimed is:

1. A compound of formula (I): 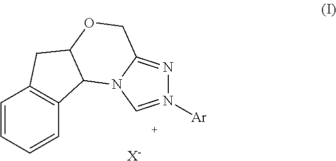
wherein Ar is:
(1) a phenyl substituted with:
(a) one or more halogens, wherein the halogen is selected from 1 to 4 fluorine atoms, 1 to 5 chlorine atoms, 1 to 5 bromine atoms, and 1 to 5 iodine atoms; or
(b) a substituent selected from the group consisting of R(Z)ₙ, NO₂, and Z, wherein R can be a substituted or unsubstituted branched or straight chain alkyl, Z can be a halogen or hydrogen, and n is 1-3; or
(2) an unsubstituted or substituted naphthyl, pyridyl, pyrymidinyl, furyl, thiophene, quinoline, or pyrrolyl;
and wherein X⁻ is a counter ion.

2. The compound of claim 1 wherein the counter ion X⁻ is selected from the group consisting of BF₄, Cl, PF₆, BPh₄, and RBF₃.

3. A composition comprising a compound of formula (I) according to claim 1, a proton donor, and a base.

4. A method for asymmetric hydration of an activated aldehyde comprising contacting the aldehyde with a proton donor, a base, and an asymmetric hydration catalyst of formula (I): 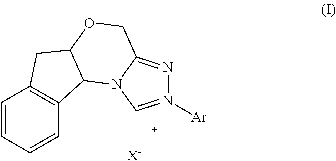
wherein Ar:
(1) a phenyl substituted with:
(a) one or more halogens, wherein the halogen is selected from 1 to 4 fluorine atoms, 1 to 5 chlorine atoms, 1 to 5 bromine atoms, and 1 to 5 iodine atoms; or
(b) a substituent selected from the group consisting of R(Z)ₙ, NO₂, and Z, wherein R can be a substituted or unsubstituted branched or straight chain alkyl, Z can be a halogen or hydrogen, and n is 1-3; or
(2) an unsubstituted or substituted naphthyl, pyridyl, pyrymidinyl, furyl, thiophene, quinoline, or pyrrolyl;
and wherein X⁻ is a counter ion;
and wherein the aldehyde undergoes asymmetric hydration to form a respective carboxylic acid of the aldehyde.

5. The method of claim 4, wherein the aldehyde is an enal.

6. The method of claim 4, wherein the aldehyde is a α,α-dichloro aldehyde or an α-chloro α-fluoro aldehyde.

7. The method of claim 4, wherein the aldehyde is selected from the group consisting of:

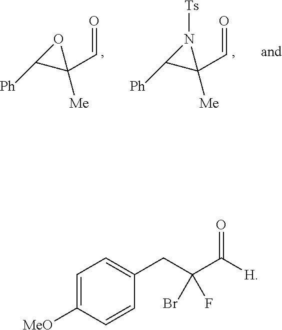

8. The method of claim 4 wherein, the asymmetric hydration results in an enantiomeric excess of the respective α-deuterio carboxylic acid, α-deuterio-α-chloro carboxylic acid or α-deuterio-α-fluoro carboxylic acid.

9. The method of claim 4 further comprising contacting the aldehyde with an additive selected from phase transfer reagents, salts, and brine.

10. A method for asymmetric hydration of a drug analog that contains an aldehyde group, the method comprising contacting the drug analog with a proton donor, a base, and an asymmetric hydration catalyst of formula (I): 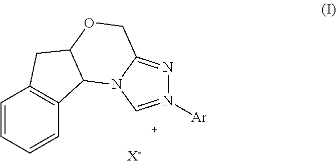
wherein Ar is:
(1) a phenyl substituted with:
(a) one or more halogens, wherein the halogen is selected from 1 to 4 fluorine atoms, 1 to 5 chlorine atoms, 1 to 5 bromine atoms, and 1 to 5 iodine atoms; or
(b) a substituent selected from the group consisting of R(Z)ₙ, NO₂, and Z, wherein R can be a substituted or unsubstituted branched or straight chain alkyl, Z can be a halogen or hydrogen, and n is 1-3; or
(2) an unsubstituted or substituted naphthyl, pyridyl, pyrymidinyl, furyl, thiophene, quinoline, or pyrrolyl;
and wherein X⁻ is a counter ion;
and wherein the drug analog comprises at least one aldehyde for asymmetric hydration to form the acid of the drug analog.

11. The method of claim 10, wherein the aldehyde is an enal.

12. The method of claim 10, wherein the aldehyde is a α,α-dichloro aldehyde or an α-chloro α-fluoro aldehyde.

13. The method of claim 10, wherein the aldehyde is selected from the group consisting of:

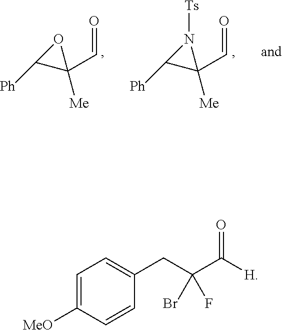

14. The method of claim 10, wherein the drug analog is an α-fluoroenal and the asymmetric hydration forms an α-fluoro carboxylic acid.

15. The method of claim 10, wherein the asymmetric hydration results in an enantiomeric excess of the respective drug analog.

16. A method for asymmetric incorporation of an α-deuterium in an activated aldehyde comprising contacting the aldehyde with D₂O and an asymmetric hydration catalyst of formula (I): 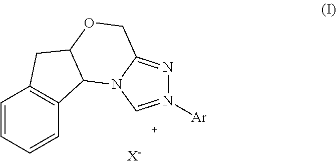
wherein Ar is:
(1) a phenyl substituted with:
(a) one or more halogens, wherein the halogen is selected from 1 to 4 fluorine atoms, 1 to 5 chlorine atoms, 1 to 5 bromine atoms, and 1 to 5 iodine atoms; or
(b) a substituent selected from the group consisting of R(Z)ₙ, NO₂, and Z, wherein R can be a substituted or unsubstituted branched or straight chain alkyl, Z can be a halogen or hydrogen, and n is 1-3; or
(2) an unsubstituted or substituted naphthyl, pyridyl, pyrymidinyl, furyl, thiophene, quinoline, or pyrrolyl;
and wherein X⁻ is a counter ion;
and wherein the aldehyde incorporates an α-deuterium.

17. The method of claim 16, wherein the aldehyde is an enal.

18. The method of claim 16, wherein the aldehyde is a α,α-dichloro aldehyde or an α-chloro α-fluoro aldehyde.

19. The method of claim 16, wherein the aldehyde is selected from the group consisting of:

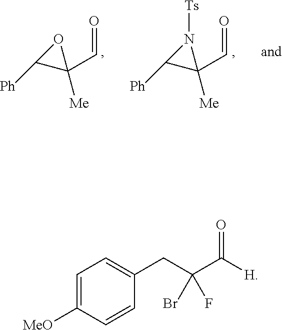

20. The method of claim 4, wherein the base is selected from the group consisting of K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaHCO₃, KHCO₃, CsHCO₃, K₂HPO₄, KH₂PO₄, KOAc, and NaOAc.

21. The method of claim 10, wherein the base is selected from the group consisting of K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaHCO₃, KHCO₃, CsHCO₃, K₂HPO₄, KH₂PO₄, KOAc, and NaOAc.

22. The method of claim 16, wherein the base is selected from the group consisting of K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaHCO₃, KHCO₃, CsHCO₃, K₂HPO₄, KH₂PO₄, KOAc, and NaOAc.

23. The method of claim 16, further comprising contacting the aldehyde with an additive selected from phase transfer reagents, salts, and brines.
